(19) United States
(12) Patent Application Publication
(10) Pub. No.: US 2022/0378786 A1
(43) Pub. Date: Dec. 1, 2022

(54) METHODS OF DEFINING FUNCTIONAL CHANGE AND SLOWING PROGRESSION IN CHRONIC LIVER DISEASE

(71) Applicant: HepQuant, LLC, Denver, CO (US)

(72) Inventor: Gregory Thomas Everson, Englewood, CO (US)

(21) Appl. No.: 17/749,491

(22) Filed: May 20, 2022

Related U.S. Application Data
(60) Provisional application No. 63/191,826, filed on May 21, 2021.

(51) Int. Cl. A61K 31/505; A61K 31/366; A61K 31/22; A61K 31/405; A61K 31/4418; A61K 31/40; A61K 31/47; A61K 31/155; A61K 31/355; A61K 31/64; A61K 31/575; A61P 1/16

(52) U.S. Cl. CPC A61K 31/505; A61K 31/366; A61K 31/22; A61K 31/405; A61K 31/4418; A61K 31/40; A61K 31/47; A61K 31/155; A61K 31/355; A61K 31/64; A61K 31/575; A61P 1/16

(57) ABSTRACT

Methods and compositions are provided for treatment of chronic liver diseases such as NASH, for example, in order to slow disease progression, and reduce portal-systemic shunting. Methods comprising cholate liver function tests and administration of a statin and a biguanide are provided. Compositions comprising a statin and a biguanide are also provided.

### CROSS-REFERENCES TO RELATED APPLICATIONS

This application claims the benefit of priority to U.S. Provisional Application Ser. No. 63/191,826, filed May 21, 2021, which is incorporated by reference herein in its entirety.

### BACKGROUND

One-third of the US population has non-alcoholic fatty liver disease (NAFLD) due to obesity and ˜8 million of these individuals have a progressive form of the disease, non-alcoholic steatohepatitis (NASH). Currently, there are few noninvasive ways to determine which individuals with NAFLD will develop NASH. This is of medical importance since NASH can be a prelude to the development of end-stage liver disease. Improved methods for prognosis and treatment of chronic liver diseases such as NASH are desirable.

### SUMMARY

Methods and compositions are provided for treatment of chronic liver diseases such as NASH, for example, in order to slow disease progression, and reduce portal-systemic shunting. Methods comprising obtaining cholate liver function test values and administration of a statin and a biguanide are provided. Compositions comprising a statin and a biguanide are also provided.

One aspect of the disclosure relates to a method of treating a chronic liver disease or disorder described herein in a subject in need thereof, comprising administering to the subject a therapeutically effective amount of a statin or a pharmaceutically acceptable salt thereof, and coadministering a therapeutically effective amount of a biguanide or a pharmaceutically acceptable salt thereof. The method may further comprise coadministering an additional agent selected from the group consisting of a vitamin E, an ursodeoxycholic acid or pharmaceutically acceptable salt thereof.

Another aspect of the disclosure relates to a method of treating NAFLD in a subject in need thereof, comprising administering to the subject a therapeutically effective amount of a statin or a pharmaceutically acceptable salt thereof, and coadministering a therapeutically effective amount of a biguanide or a pharmaceutically acceptable salt thereof. The method may further comprise coadministering an additional agent selected from the group consisting of a vitamin E, an ursodeoxycholic acid or pharmaceutically acceptable salt thereof.

Another aspect of the disclosure relates to a method of treating NASH in a subject in need thereof, comprising administering to the subject a therapeutically effective amount of a statin or a pharmaceutically acceptable salt thereof, and coadministering a therapeutically effective amount of a biguanide or a pharmaceutically acceptable salt thereof. The method may further comprise coadministering an additional agent selected from the group consisting of a vitamin E, an ursodeoxycholic acid or pharmaceutically acceptable salt thereof.

Another aspect of the disclosure relates to a method of slowing down or reversing the progression of NASH in a subject in need thereof, comprising administering to the subject a therapeutically effective amount of a statin or a pharmaceutically acceptable salt thereof, and coadministering a therapeutically effective amount of a biguanide or a pharmaceutically acceptable salt thereof. The method may further comprise coadministering an additional agent selected from the group consisting of a vitamin E, an ursodeoxycholic acid or pharmaceutically acceptable salt thereof.

Another aspect of the disclosure relates to a method of slowing down or reversing the progression of liver fibrosis in a subject in need thereof, comprising administering to the subject a therapeutically effective amount of a statin or a pharmaceutically acceptable salt thereof, and coadministering a therapeutically effective amount of a biguanide or a pharmaceutically acceptable salt thereof. The method may further comprise coadministering an additional agent selected from the group consisting of a vitamin E, an ursodeoxycholic acid or pharmaceutically acceptable salt thereof.

Another aspect of the disclosure relates to a method of slowing down or reversing the progression of cirrhosis (e.g., compensated cirrhosis) in a subject in need thereof, comprising administering to the subject a therapeutically effective amount of a statin or a pharmaceutically acceptable salt thereof, and coadministering a therapeutically effective amount of a biguanide or a pharmaceutically acceptable salt thereof. The method may further comprise coadministering an additional agent selected from the group consisting of a vitamin E, an ursodeoxycholic acid or pharmaceutically acceptable salt thereof.

Another aspect of the disclosure relates to use of a statin or a pharmaceutically acceptable salt thereof, in combination with a biguanide or a pharmaceutically acceptable salt thereof, for treating NAFLD, treating NASH, slowing down or reversing the progression of NASH, slowing down or reversing the progression of liver fibrosis, and/or slowing down or reversing the progression of cirrhosis (e.g., compensated cirrhosis).

Another aspect of the disclosure relates to a statin or a pharmaceutically acceptable salt thereof, in combination with a biguanide or a pharmaceutically acceptable salt thereof for use in treating NAFLD, treating NASH, slowing down or reversing the progression of NASH, slowing down or reversing the progression of liver fibrosis, and/or slowing down or reversing the progression of cirrhosis (e.g., compensated cirrhosis).

Another aspect of the disclosure relates to use of a statin or a pharmaceutically acceptable salt thereof, in combination with a biguanide or a pharmaceutically acceptable salt thereof, in the manufacture of a medicament for treating NAFLD, treating NASH, slowing down or reversing the progression of NASH, slowing down or reversing the progression of liver fibrosis, and/or slowing down or reversing the progression of cirrhosis (e.g., compensated cirrhosis).

Another aspect of the disclosure relates to a statin or a pharmaceutically acceptable salt thereof, in combination with a biguanide or a pharmaceutically acceptable salt thereof for use in the manufacture of a medicament for treating NAFLD, treating NASH, slowing down or reversing the progression of NASH, slowing down or reversing the progression of liver fibrosis (e.g., NASH with fibrosis), and/or slowing down or reversing the progression of cirrhosis (e.g., compensated cirrhosis due to NASH).

A method of treating or preventing a chronic liver disease or disorder in a subject is provided comprising:

a) determining a first liver function test value in the subject at a time point prior to the administration of a therapeutically effective amount of a statin or a pharmaceutically acceptable salt thereof, and coadministering a therapeutically effective amount of a biguanide or a pharmaceutically acceptable salt thereof;

b) administering to the subject a therapeutically effective amount of a statin or a pharmaceutically acceptable salt thereof, and a therapeutically effective amount of a biguanide or a pharmaceutically acceptable salt thereof;

c) determining at least a second liver function test value in the subject at least one time point after the administration of the therapeutically effective amount of a statin or a pharmaceutically acceptable salt thereof, and a therapeutically effective amount of a biguanide or a pharmaceutically acceptable salt thereof;

d) determining a liver function test difference score by calculating the difference between the at least second liver function test value and the first liver function test value;

e) comparing the liver function test difference score to a predetermined cutoff value.

In some embodiments, the method of treating or preventing a chronic liver disease or disorder further comprises:

f) discontinuing or decreasing administration of the statin or a pharmaceutically acceptable salt thereof, and coadministration of the biguanide or a pharmaceutically acceptable salt thereof when the liver function difference score is less than or equal to the predetermined cutoff value, or continuing administration of the statin or a pharmaceutically acceptable salt thereof, and the biguanide or a pharmaceutically acceptable salt thereof when the liver function difference score is greater than or equal to the predetermined cutoff value.

The liver function test difference score may be used to evaluate hepatic function in the subject over time, to determine treatment efficacy, to increase or decrease the dosage of the statin or pharmaceutically acceptable salt thereof, to increase or decrease the dosage of the biguanide or pharmaceutically acceptable salt thereof, to discontinue the administration of the statin or pharmaceutically acceptable salt thereof, to discontinue the administering of the biguanide or pharmaceutically acceptable salt thereof, or to indicate an additional agent should be coadministered. The additional agent may be selected from the group consisting of a vitamin E, ursodeoxycholic acid, sulfonylurea, and PPAR-gamma agonist.

The first liver function test value may be used to determine baseline hepatic function in the subject, determine a need for treatment in the subject, indicate disease progression of any chronic liver disease, indicate disease severity of any chronic liver disease, indicate frequency of obtaining the at least second liver function test value, or indicate that an additional liver function test should be obtained. The frequency of repeating the liver function test may be weekly, bimonthly, monthly, biannually, annually, every two years, every three years, every five years, or every ten years.

The chronic liver disease or disorder may be selected from the group consisting of non-alcoholic steatohepatitis (NASH), non-alcoholic fatty liver disease (NAFLD), alcoholic liver disease, drug-induced liver disease, chronic hepatitis C, chronic hepatitis B, cytomegalovirus, Epstein Barr virus, portal hypertension, cryptogenic cirrhosis, alpha 1-antitrypsin disease, hemochromatosis, nodular regenerative hyperplasia, idiopathic liver disease, congenital liver diseases, haemochromatosis, Wilson's disease, autoimmune chronic hepatitis, primary biliary cirrhosis, primary sclerosing cholangitis (PSC), liver damage due to progressive fibrosis, liver fibrosis, and hepatocellular carcinoma (HCC). The subject may be suffering from diabetes mellitus (DM).

The liver function test may be a cholate liver function test. For example, the cholate liver function test may be selected from the group consisting of cholate SHUNT fraction, DSI value, portal hepatic filtration rate (portal HFR)(mL min⁻¹kg⁻¹), systemic hepatic filtration rate (systemic HFR)(mL min⁻¹kg⁻¹), indexed Hepatic Reserve (HRindex), STAT value (uM), k_{FP elim} (min⁻¹), IV clearance (mL min⁻¹), PO clearance (ml min⁻¹), and RCA-20 (fraction retained).

The statin may be any suitable statin approved for human use. For example, the statin may be selected from the group consisting of lovastatin, pravastatin, simvastatin, fluvastatin, cerivastatin, atorvastatin, pitavastatin, and rosuvastatin, or a pharmaceutically acceptable salt thereof. The daily dose of the statin may be within a range from about 5 mg to about 100 mg daily, about 10 mg to about 80 mg daily, or about 20 mg to about 40 mg.

The biguanide may be any suitable biguanide approved for human use. The biguanide may be metformin or a pharmaceutically acceptable salt thereof. The daily dose of the biguanide may be in a range from about 500 mg to about 2550 mg daily, about 800 to about 2,000 mg daily, or about 1,000 mg/daily to about 1,500 mg.

The method may further comprise coadministration of an additional agent selected from the group consisting of vitamin E, ursodeoxycholic acid, sulfonylurea, and PPAR-gamma agonist.

A method of treating a chronic liver disease in a subject is provided comprising administering a statin and a biguanide. Optionally a vitamin E and/or ursodeoxycholic acid may be coadministered. The chronic liver disease may be selected from the group consisting of nonalcoholic fatty liver disease (NAFLD), nonalcoholic steatohepatitis (NASH), hepatitis B infection, hepatitis C infection, alcoholic liver disease, liver damage due to progressive fibrosis, or liver fibrosis.

In some embodiments, the liver disease is NASH.

In some embodiments, the subject suffers from diabetes mellitus (DM).

A pharmaceutical composition is provided comprising a statin or a pharmaceutically acceptable salt thereof, a biguanide or a pharmaceutically acceptable salt thereof, and a pharmaceutically acceptable carrier or excipient.

The pharmaceutical composition may comprise a statin or pharmaceutically acceptable salt thereof selected from the group consisting of lovastatin, pravastatin, simvastatin, fluvastatin, cerivastatin, atorvastatin, and rosuvastatin, or a pharmaceutically acceptable salt thereof. The pharmaceutical composition may comprise the statin or pharmaceutically acceptable salt thereof in a range of from about 5 mg to about 100 mg.

The pharmaceutical composition may comprise metformin or a pharmaceutically acceptable salt thereof. The pharmaceutical composition may comprise the metformin or pharmaceutically acceptable salt thereof in a range from about 250 mg to about 2500 mg.

The pharmaceutical composition may comprise from about 5 mg to about 100 mg of the statin or pharmaceutically acceptable salt thereof and about 250 mg to about 2500 mg daily of the biguanide or pharmaceutically acceptable salt thereof.

A method of treating or preventing a chronic liver disease or disorder in a subject in need thereof is provided comprising administering a composition comprising a statin or a pharmaceutically acceptable salt thereof, a biguanide or a pharmaceutically acceptable salt thereof, and a pharmaceutically acceptable carrier or excipient.

### BRIEF DESCRIPTION OF THE DRAWINGS

FIG. 1 shows a flow diagram of numbers of subjects with and without NASH and with and without diabetes mellitus (DM) in the SHUNT-V clinical study. The distribution of NASH and diabetes in 270 subjects evaluated for analysis of impact of NASH and diabetes on clinical scores (CP, MELD, lab results), transient and shear-wave elastography, endoscopic findings, and HepQuant cholate liver function tests.

FIG. 2 shows Table 1 with demographic information for 270 subjects with mean age, sex, weight (kg), height (cm), body mass index (BMI), and rate of obesity (BMI>30) sorted for analysis as to NASH and non-NASH and DM and non-DM groups. The NASH and diabetic patients were found to be older, heavier, and more likely to be obese.

FIG. 3 shows Table 2 with clinical information for 270 subjects showing mean Child-Pugh (CP) score (±SD), mean MELD score (±SD), mean total bilirubin (±SD), avg. INR (International Normalized Ratio prothrombin time) (±SD), and avg. creatinine (±SD) sorted for analysis as to NASH and non-NASH and DM and non-DM groups and. Surprisingly, Child-Pugh (CP) score and bilirubin trended lower in NASH and diabetic patients.

FIG. 4 shows Table 3 with elastography data for NASH (n=50) vs. non-NASH (n=34) subjects and diabetes (n=51) vs non-diabetes (n=33) subjects. Data includes liver stiffness measure (LSM, kPa) (±SD) as determined by transient elastography (TE) or shear-wave elastography (SW), percent of subjects exhibiting <20 kPa LSM, percent of fibrosis score F4 subjects (advanced liver scarring cirrhosis) (highest medical need population), and avg. CAP score (±SD). Liver stiffness (LSM, kPa) and CAP scores were similar in NASH vs. non-NASH and Diabetes vs. No Diabetes subjects.

FIG. 5 shows Table 4 with endoscopic data for 270 subjects sorted between NASH vs. non-NASH subjects and diabetes vs. no diabetes subjects. Numbers of subjects having any varices, esophageal small varices, large varices, red wale sign, gastric varices, any portal hypertensive gastropathy (PHG), moderate to severe PHG, and endoscopic evidence for significant portal hypertension (increase in pressure within the portal vein). Endoscopic findings were similar between NASH vs. non-NASH and diabetes vs. no diabetes subjects.

FIG. 6 shows Table 5 with HepQuant cholate liver function test values for 270 subjects sorted between NASH vs. non-NASH subjects and diabetes vs. no diabetes. Mean values (±SD) for cholate elimination rate, kelim (min⁻¹), cholate volume of distribution (Vd), (L kg-1), RCA20, IV Cl (ml min⁻¹), systemic HFR (ml min⁻¹ kg⁻¹), PO Cl (mL min-1), portal HFR (mL min-1 kg-1), STAT (uM), SHUNT (%), DSI, and HRindexed are shown. Surprisingly, in the SHUNT-V cohort, NASH and especially Diabetes were associated with better liver function and less portal-systemic shunting, compared to the non-NASH and no diabetes subjects.

FIG. 7A shows a bar graph with systemic HFR data for NASH vs. non-NASH subjects further sorted into NASH and diabetes mellitus (DM), NASH with no DM, Non-NASH with DM and non-NASH no DM subjects. Data for NASH vs. non-NASH subjects was not found to be significantly different.

FIG. 7B shows a bar graph with systemic HFR data for Diabetes vs. no-Diabetes subjects further sorted into NASH and diabetes mellitus (DM), NASH with no DM, Non-NASH with DM and non-NASH no DM subjects. Mean systemic HFR was significantly higher in diabetes subjects compared to non-diabetes subjects (p=0.0325).

FIG. 8A shows a bar graph with portal HFR data for NASH vs. non-NASH subjects further sorted into NASH and diabetes mellitus (DM), NASH with no DM, Non-NASH with DM and non-NASH no DM subjects. Mean portal HFR trended higher in NASH vs. non-NASH subjects (p=0.06).

FIG. 8B shows a bar graph with portal HFR data for Diabetes vs. no-Diabetes subjects further sorted into NASH and diabetes mellitus (DM), NASH with no DM, Non-NASH with DM and non-NASH no DM subjects. Mean portal HFR was significantly higher in diabetes subjects vs. no diabetes subjects (p=0.0004).

FIG. 9A shows a bar graph with cholate SHUNT fraction data for NASH vs. non-NASH subjects further sorted into NASH and diabetes mellitus (DM), NASH with no DM, Non-NASH with DM and non-NASH no DM subjects. Mean SHUNT fraction was significantly lower in NASH vs. non-NASH subjects (p=0.0256).

FIG. 9B shows a bar graph with cholate SHUNT fraction data for Diabetes vs. no-Diabetes subjects further sorted into NASH and diabetes mellitus (DM), NASH with no DM, Non-NASH with DM and non-NASH no DM subjects. Mean SHUNT fraction was significantly lower in diabetes subjects vs. no diabetes subjects (p=0.0013).

FIG. 10A shows a bar graph with DSI data for NASH vs. non-NASH subjects further sorted into NASH and diabetes mellitus (DM), NASH with no DM, Non-NASH with DM and non-NASH no DM subjects. Mean DSI value was significantly lower in NASH vs. non-NASH subjects (p=0.0375).

FIG. 10B shows a bar graph with DSI data for Diabetes vs. no-Diabetes subjects further sorted into NASH and diabetes mellitus (DM), NASH with no DM, Non-NASH with DM and non-NASH no DM subjects. Mean DSI value was significantly lower in diabetes subjects vs. no diabetes subjects (p=0.0008).

FIG. 11A shows a bar graph with indexed Hepatic Reserve (HRindexed) data for NASH vs. non-NASH subjects further sorted into NASH and diabetes mellitus (DM), NASH with no DM, Non-NASH with DM and non-NASH no DM subjects. Mean indexed Hepatic Reserve (HRindexed) was significantly higher in NASH vs. non-NASH subjects (p=0.0383).

FIG. 11B shows a bar graph with indexed Hepatic Reserve (HRindexed) data for Diabetes vs. no-Diabetes subjects further sorted into NASH and diabetes mellitus (DM), NASH with no DM, Non-NASH with DM and non-NASH no DM subjects. Mean indexed Hepatic Reserve (HRindexed) was significantly higher in diabetes subjects vs. no diabetes subjects (p=0.0008).

FIG. 12A shows a bar graph with cholate STAT data for NASH vs. non-NASH subjects further sorted into NASH and diabetes mellitus (DM), NASH with no DM, Non-NASH with DM and non-NASH no DM subjects. Mean cholate STAT value was significantly lower in NASH vs. non-NASH subjects (p=0.0312).

FIG. 12B shows a bar graph with cholate STAT data for Diabetes vs. no-Diabetes subjects further sorted into NASH and diabetes mellitus (DM), NASH with no DM, Non-NASH with DM and non-NASH no DM subjects. Mean cholate STAT value was significantly lower in diabetes subjects vs. no diabetes subjects (p=0.0004).

FIG. 13A shows a bar graph with kFP elim data for NASH vs. non-NASH subjects further sorted into NASH and diabetes mellitus (DM), NASH with no DM, Non-NASH with DM and non-NASH no DM subjects. Mean Cholate Elimination Rate, kₑₗᵢₘ min⁻¹(kFP elim; min⁻¹) was significantly higher in NASH vs. non-NASH subjects (p=0.0230).

FIG. 13B shows a bar graph with kFP elim data for Diabetes vs. no-Diabetes subjects further sorted into NASH and diabetes mellitus (DM), NASH with no DM, Non-NASH with DM and non-NASH no DM subjects. Mean Cholate Elimination Rate, kₑₗᵢₘ min⁻¹(kFP elim; min⁻¹) was significantly higher in diabetes subjects vs. no diabetes subjects (p=0.0045).

FIG. 14 shows a bar graph with mean cholate volume of distribution (Vd; L kg⁻¹) sorted into NASH and diabetes mellitus (DM), NASH with no DM, Non-NASH with DM and non-NASH no DM subjects. No significant difference in Vd between NASH vs. non-NASH subjects or between diabetes vs. no diabetes subjects was observed.

FIG. 15A shows a bar graph with intravenous cholate clearance (IV Cl)(ml min⁻¹) data for NASH vs. non-NASH subjects further sorted into NASH and diabetes mellitus (DM), NASH with no DM, Non-NASH with DM and non-NASH no DM subjects. Mean intravenous cholate clearance (IV Cl) (mL min⁻¹) was significantly higher in NASH vs. non-NASH subjects (p=0.0081).

FIG. 15B shows a bar graph with intravenous cholate clearance (IV Cl)(ml min⁻¹) data for Diabetes vs. no Diabetes subjects further sorted into NASH and diabetes mellitus (DM), NASH with no DM, Non-NASH with DM and non-NASH no DM subjects. Mean intravenous cholate clearance (IV Cl) (mL min⁻¹) was significantly higher in diabetes subjects vs. no diabetes subjects (p=0.0004).

FIG. 16A shows a bar graph with oral cholate clearance (PO Cl)(ml min⁻¹) data for NASH vs. non-NASH subjects further sorted into NASH and diabetes mellitus (DM), NASH with no DM, Non-NASH with DM and non-NASH no DM subjects. Mean oral cholate clearance (PO Cl) (mL min⁻¹) was significantly higher in NASH vs. non-NASH subjects (p=0.0030).

FIG. 16B shows a bar graph with oral cholate clearance (PO Cl)(ml min⁻¹) data for Diabetes vs. no Diabetes subjects further sorted into NASH and diabetes mellitus (DM), NASH with no DM, Non-NASH with DM and non-NASH no DM subjects. Mean oral cholate clearance (PO Cl) (mL min⁻¹) was significantly higher in diabetes subjects vs. no diabetes subjects (p<0.0001).

FIG. 17A shows a bar graph with RCA-20 data for NASH vs. non-NASH subjects further sorted into NASH and diabetes mellitus (DM), NASH with no DM, Non-NASH with DM and non-NASH no DM subjects. Mean RCA-20 (fraction retained) (%) was significantly lower in NASH vs. non-NASH subjects (p=0.0171).

FIG. 17B shows a bar graph with RCA-20 data for Diabetes vs. no Diabetes subjects further sorted into NASH and diabetes mellitus (DM), NASH with no DM, Non-NASH with DM and non-NASH no DM subjects. Mean RCA-20 (fraction retained) (%) was significantly lower in diabetes subjects vs. no diabetes subjects (p=0.0106).

FIG. 18 shows Table 6 with data from SHUNT-V study (ON-OFF drug) with DSI and SHUNT results relative to concomitant medications. Data for a subset of 130 subjects taking anti-diabetic drug therapies and vitamins, insulin, metformin, glipizide, pioglitazone, GLP-1 analogues, SGLT2 analogues, or statins, and data for a subset of 140 subjects not taking anti-diabetic therapies taking statins (n=25) or not taking statins (n=115). Mean difference in DSI, and mean difference in SHUNT values are shown. Surprisingly, in subjects taking both anti-diabetic drug therapies and a statin, the mean difference in DSI was −3.6 (p=0.0044) and the mean difference in SHUNT was −7.6% (p=0.0141) indicating significantly better liver function and decreased portal-systemic shunting.

FIG. 19 shows Table 7 with endoscopic EGD findings for subjects having NASH with diabetes (n=82), NASH without diabetes (n=41), Non-NASH with diabetes (n=33), and Non-NASH without diabetes (n=114). Number of subjects in each group having any varices, esophageal small varices, large varices, red wale sign, gastric varices, any portal hypertensive gastropathy (PHG), moderate to severe PHG, and endoscopic evidence for significant portal hypertension (increase in pressure within the portal vein). Endoscopic findings were similar between NASH vs. non-NASH and diabetes vs. no diabetes subjects.

FIG. 20 shows Table 8 with HepQuant cholate liver function test results for subjects having NASH with diabetes (n=82), NASH without diabetes (n=41), Non-NASH with diabetes (n=33), and Non-NASH without diabetes (n=114). Surprisingly NASH and Diabetes subjects exhibited significantly better liver function and less portal-systemic shunting in the cholate liver function tests compared to the non-NASH and no diabetes subjects in a number of HepQuant tests.

FIG. 21 shows Table 9 with elastography findings within 1 year of enrollment was performed in 53 of 123 NASH subjects (43%) and 36 of 147 non-NASH subjects (24%). CAP scores for non-NASH subjects, with diabetes vs. no diabetes exhibited a trend to lower CAP scores (p=0.0782). A lower CAP score indicates a trend to lower amount of hepatic steatosis.

FIG. 22 shows a bar graph of the effect of diabetic and/or lipid lowering drugs in SHUNT % score. Combined diabetic drug and lipid-lowering drug use is associated with significantly less portal-systemic shunting as indicated by lower SHUNT %. *p<0.0001 value is for change from treatment with neither drug class to both classes of drug.

FIG. 23 shows a bar graph of the effect of diabetic and/or lipid lowering drugs in DSI score. Combined diabetic drug and lipid-lowering drug use is associated with substantially improved liver function as indicated by significantly lower DSI scores. *p<0.0001 value is for change from treatment with neither drug class to both classes of drug.

### DETAILED DESCRIPTION OF THE INVENTION

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “about,” when referring to a measurable value such as an amount of a compound, dose, time, temperature, and the like, is meant to encompass variations of 10%, 5%, 1%, 0.5%, or even 0.1% of the specified amount.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs. In the event of conflicting terminology, the present specification is controlling.

All patents, patent applications and publications referred to herein are incorporated by reference in their entirety.

A “subject” or “patient” includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). In one embodiment, the patient is human. In one embodiment, the subject is human child (e.g., between about 30 kg to about 70 kg).

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

Oral compositions may include a pharmaceutically acceptable carrier, excipient, binder, diluent, disintegrating agent, lubricant, sweetening agent, and/or flavoring. Pharmaceutically acceptable carriers may be used to protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The oral or parenteral compositions may be formulated in a dosage unit form for ease of administration and uniformity of dosage. Dosage unit form as used herein refers to physically discrete units suited as unitary dosages for the subject to be treated; each unit containing a predetermined quantity of drug or drug particles calculated to produce the desired therapeutic effect in association with the required pharmaceutical carrier. The specification for the dosage unit forms of the disclosure are dictated by and directly dependent on the unique characteristics of the statin and biguanide drugs and the particular therapeutic effect to be achieved.

The compositions may be enclosed in gelatin capsules or compressed into tablets. For the purpose of oral therapeutic administration, the active compounds comprising a statin and a biguanide, may be incorporated with carriers and/or excipients and used in the form of tablets, troches, or capsules. Pharmaceutically acceptable binding agents, and/or adjuvant materials may be included as part of the composition. The tablets, pills, capsules, troches and the like can contain any of the following ingredients, or compounds of a similar nature: a binder such as microcrystalline cellulose, gum tragacanth or gelatin; an excipient such as sodium starch glycolate, starch or lactose, a diluent such as microcrystalline cellulose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

A pharmaceutical composition of the disclosure may be formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A “control” as used herein refers to a baseline level determined on a patient-by-patient basis, an amount or level considered by those skilled in the art as a normal value, or any level or measure of a condition or biomarker described herein taken from a patient or population of patients at any given time for a given condition.

The term “Fibrosis” refers to a condition involving the development of excessive fibrous connective tissue, e.g., scar tissue, in a tissue or organ. Such generation of scar tissue may occur in response to infection, inflammation, or injury of the organ due to a disease, trauma, chemical toxicity, and so on. Fibrosis may develop in a variety of different tissues and organs, including the liver, kidney, intestine, lung, heart, etc.

The term “Cirrhosis” refers to a condition in which the liver is scarred and permanently damaged. Scar tissue replaces healthy liver tissue and prevents the liver from working normally. As cirrhosis gets worse, the liver begins to fail. Compensated cirrhosis often does not exhibit signs or symptoms related to cirrhosis, despite evidence of portal hypertension, such as esophageal or gastric varices. In contrast, decompensated cirrhosis displays symptomatic complications related to cirrhosis, including those related to hepatic insufficiency (jaundice or hepatic encephalopathy), and those related to portal hypertension (ascites or variceal hemorrhage). Prognosis and survival is markedly better in patients with compensated cirrhosis than in those with decompensated cirrhosis. In addition, the presence of decompensated cirrhosis can have major implications regarding management and prevention of cirrhosis-related complications, as well as consideration for a referral for liver transplantation evaluation.

The term “treating” includes any effect, e.g., lessening, reducing, modulating, or eliminating, that results in the improvement of the condition, disease, disorder, etc. “Treating” or “treatment” of a disease state includes: arresting the development of the disease state or its clinical symptoms; or relieving the disease state, i.e., causing temporary or permanent regression of the disease state or its clinical symptoms.

The term “preventing” the disease state includes causing the clinical symptoms of the disease state not to develop in a subject that may be exposed to or predisposed to the disease state, but does not yet experience or display symptoms of the disease state.

The term “inhibiting” or “inhibition,” as used herein, refers to any detectable positive effect on the development or progression of a disease or condition. Such a positive effect may include the delay or prevention of the onset of at least one symptom or sign of the disease or condition, alleviation or reversal of the symptom(s) or sign(s), and slowing or prevention of the further worsening of the symptom(s) or sign(s).

The term “disease state” means any disease, disorder, condition, symptom, or indication.

The term “effective amount” as used herein refers to an amount of statin and biguanide that produces an acute or chronic therapeutic effect upon appropriate dose administration. The effect includes the prevention, correction, inhibition, or reversal of the symptoms, signs and underlying pathology of a disease/condition (e.g., fibrosis of the liver, kidney, or intestine) and related complications to any detectable extent.

A “therapeutically effective amount” means the amount of drug that, when administered to a mammal for treating a disease, is sufficient to effect such treatment for the disease. The “therapeutically effective amount” will vary depending on the drug, drug combination, the disease and its severity and the age, weight, etc., of the mammal to be treated. A therapeutically effective amount can refer to a starting dose or adjusted dose as set forth herein. A therapeutically effective amount of a drug can be formulated with a pharmaceutically acceptable carrier for administration to a human or an animal. Accordingly, a statin or biguanide or its formulations can be administered, for example, via oral, parenteral, or topical routes, to provide an effective amount of the compound.

For any compound, the therapeutically effective amount can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration may be adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

A “starting dose” as used herein refers to an initial dose provided to a patient to provide a clinical effect while minimizing onset or occurrence of an adverse effect. A starting dose may be less than an amount typically administered to a patient. A starting dose is provided in an amount that is titrated or gradually increased over the course of a titration period or during the course of treatment with a pharmaceutical composition described herein.

The term “administering” refers to the act of delivering an pharmaceutical composition described herein into a subject by such routes as oral, mucosal, topical, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration. The term can also refer to the frequency (e.g., daily, weekly, monthly, etc.) of providing an pharmaceutical composition described herein to a patient. Administration generally occurs after the onset of the disease, disorder, or condition, or its symptoms but, in certain instances, can occur before the onset of the disease, disorder, or condition, or its symptoms (e.g., administration for patients prone to such a disease, disorder, or condition). In certain embodiments, administration as used herein refers to oral administration.

The term “co-administration” refers to administration of two or more agents (e.g., an statin composition and a biguanide composition). The timing of co-administration depends in part of the combination and the compositions administered and can include administration at the same time, prior to, or after the administration of one or more additional therapies. An statin composition of the instant invention can be coadministered to the subject with a biguanide composition. Co-administration is meant to include simultaneous or sequential administration individually or in combination (more than one compound or agent). Thus, the preparations can also be combined, when desired, with other active substances (e.g., to reduce metabolic degradation). The pharmaceutical compositions described herein can be used in combination with each other, with other active agents known to be useful in treating a disease, for example vitamin E, ursodeoxycholic acid, a sulfonylurea, or with adjunctive agents that are not effective alone, but can contribute to or enhance the efficacy of the active agent.

“Hepatic impairment” may be used in accordance with its standard meaning(s) in the art and can, in certain embodiments herein refer to scoring based upon the Child-Pugh Score of A, B, and C.

Patients with fibrosis may be categorized in four groups: mild fibrosis (F1; mild liver scarring); moderate fibrosis (F2; moderate liver scarring); severe fibrosis (F3; severe liver scarring); and cirrhosis (F4) (advanced liver scarring).

Patients with NASH may be categorized in four groups: patients with NASH with mild fibrosis (F1) (which is largely undiagnosed and the patients may benefit from treating underlying pathology, e.g., hyperlipidemia, diabetes, obesity, lifestyle modifications); patients with NASH with moderate/severe fibrosis (F2/F3) (may benefit from treatment); and patients with NASH with cirrhosis (F4) (highest medical need population).

Elastography is a medical imaging technique that maps the elastic properties and stiffness of soft tissue. Typically diseased livers are stiffer than healthy ones. Prominent imaging techniques include use of ultrasound or magnetic resonance imaging (MRI) to determine stiffness. Liver elastography techniques may include, for example, transient elastography (TE) (e.g., FibroScan®), shear-wave elastography (SW), or magnetic resonance elastography (MRE). Elastography may be used to determine fibrosis (scarring) and steatosis (fatty change) in the liver. An LSM score (liver stiffness measurement) is reported in kPa.

A CAP score may be determined by elastography and is measured in decibels per meter (dB/m). Typical values range from 100 to 400 dB/m. The CAP score (controlled attenuation parameter) is a measurement of hepatic steatosis, or fatty change in the liver. The CAP score may be employed to determine steatosis grade. A CAP score of 238 to 260 dB/m indicates steatosis grade S1, or 11-33% amount of liver with fatty change. A CAP score of 260 to 290 dB/m indicates a steatosis grade of S2, or 34-66% amount of liver with fatty change. A CAP score higher than 290 dB/m indicates a steatosis grade of S3, or 67% or more amount of liver with fatty change.

The term “Childs-Turcotte-Pugh (CTP) score” or “Child-Pugh score” refers to a classification system used to assess the prognosis of chronic liver disease as provided in Pugh et al., Transection of the oesophagus for bleeding oesophageal varices. Br J Surg 1973; 60:646-649, which is incorporated herein by reference. The CTP score includes five clinical measures of liver disease; each measure is scored 1-3, with 3 being the most severe derangement. The five scores are added to determine the CTP score. The five clinical measures include total bilirubin, serum albumin, prothrombin time international normalized ratio (PT INR), ascites, and hepatic encephalopathy. The CTP score is one scoring system used in stratifying the seriousness of end-stage liver disease. Chronic liver disease is classified into Child-Pugh class A to C, employing the added score. Child-Pugh class A refers to CTP score of 5-6. Child-Pugh class B refers to CTP score of 7-9. Child-Pugh class C refers to CTP score of 10-15. A website calculates post-operative mortality risk in patients with cirrhosis. http://mayoclinic.org/meld/mayomodel9.html

Child-Pugh (CP) classification may be used as a prognostic indicator of hepatic impairment and cirrhosis in addition to distinguishing the disease by clinical phases. CP utilizes 2 clinical parameters (hepatic encephalopathy and ascites) and three laboratory values (bilirubin, albumin, and prothrombin time [PT]/international randomized ratio [INR]). Patients are classified as Class A (mild), Class B (moderate), or Class C (severe) based on their total CP score.

The term “Model for End-Stage Liver Disease (MELD) refers to a scoring system used to assess the severity of chronic liver disease. MELD was developed to predict death within three months of surgery in patients who had undergone a transjugular intrahepatic portosystemic shunt (TIPS) procedure patients for liver transplantation. MELD is also used to determine prognosis and prioritizing for receipt of a liver transplant. The MELD uses a patient's values for serum bilirubin, serum creatinine, and international normalized ratio for prothrombin time (INR) to predict survival. The scoring system is used by the United Network for Organ Sharing (UNOS) and Eurotransplant for prioritizing allocation of liver transplants instead of the older Child-Pugh score. See UNOS (2009 Jan. 28) “MELD/PELD calculator documentation”, which is incorporated herein by reference. For example, in interpreting the MELD score in hospitalized patients, the 3 month mortality is: 71.3% mortality for a MELD score of 40 or more.

The term “Ishak Fibrosis Score” is used in reference to a scoring system that measures the degree of fibrosis (scarring) of the liver, which is caused by chronic necroinflammation. A score of 0 represents no fibrosis, and 6 is established fibrosis. Scores of 1 and 2 indicate mild degrees of portal fibrosis; stages 3 and 4 indicate moderate (bridging) fibrosis. A score of 5 indicates nodular formation and incomplete cirrhosis, and 6 is definite cirrhosis.

Esophagogastroduodenoscopy (EGD) is an endoscopic procedure which may be used to examine the lining of the esophagus, stomach, and duodenum. EGD may be used for evaluation and/or treatment of varices, e.g., to reduce potential for bleeding. “Esophageal varices” are abnormal, dilated veins in the esophagus. A “red wale sign” is an endoscopic sign suggestive of recent hemorrhage, or propensity to bleed, seen in individuals with esophageal varices.

Liver biochemistry including ALP (alkaline phosphatase), ALT (alanine transaminase), AST (aspartate transaminase), GGT (gamma-glutamyl transpeptidase), total and conjugated bilirubin, creatinine, and albumin), prothrombin time (PT)/INR, serum electrolytes, and assessment of Child Pugh (CP) and MELD scores can be also monitored in the subject. Model for End-Stage Liver Disease (MELD) is a scoring system used to assess the severity of chronic liver disease.

Various biomarkers can be measured to determine the presence and severity of liver diseases. These biomarkers include bilirubin, albumin, and prothrombin. Bilirubin is made during normal breakdown of red blood cells. Bilirubin passes through the liver and is excreted out of the body. Bilirubin level can be measured through a blood test. Higher than normal levels of bilirubin may indicate liver problems. Albumin is a protein made by the liver. An albumin test may be ordered as part of a liver panel to evaluate liver function. The typical value for serum albumin in blood is 3.4 to 5.4 grams per deciliter. Low albumin levels can indicate a number of health conditions, including liver diseases. Prothrombin time (PT) measures how long it takes blood to form a clot, and is a universal indicator of liver disease severity. In addition, the serum level of cortisol or fibrinogen a chain may be assessed to determine liver function and the presence of liver diseases.

The liver function test may be a cholate liver function test. The liver function test may be a cholate liver function test selected from the group consisting of cholate SHUNT fraction, DSI value, portal hepatic filtration rate (portal HFR)(mL min⁻¹kg⁻¹), systemic hepatic filtration rate (systemic HFR)(mL min⁻¹kg⁻¹), indexed Hepatic Reserve (HRindex), STAT value (uM), k_{FP elim} (min⁻¹), IV clearance (mL min⁻¹), PO clearance (ml min⁻¹), and RCA-20 (fraction retained).

Briefly, DSI is a score without units representing a quantitative measurement of liver function. DSI (Disease Severity Index) is a score that is a function of the sum of cholate clearances from systemic and portal circulations adjusted to disease severity ranging from healthy persons to end-stage liver disease. Hepatic Reserve represents a percentage of maximum hepatic functional capacity measured by DSI normalized to the DSI range in persons of lean body mass. Individual Risk Score for Annual Clinical Events (RISK-ACE) may be based upon baseline DSI (Model A) and also baseline DSI plus the ΔDSI that occurred over 2 years (Model D) in an HCV population with approximately 25% experiencing clinical event over a maximum of 8.7 years of followup. SHUNT % represents a quantitative measurement of portal-systemic shunting. SHUNT % is a measurement of the percentage of spillover of the orally administered d4-cholate. The first-pass hepatic elimination of cholate in percent of orally administered cholate is defined as (100%—SHUNT). Systemic HFR, mL min⁻¹ kg⁻¹ represents a model independent clearance of intravenously injected 13C-cholate, adjusted for body weight, and calculated from dose/AUC. Portal HFR, mL min⁻¹ kg⁻¹ represents a model independent apparent clearance of orally administered d4-cholate, adjusted for body weight, and calculated from dose/AUC. Cholate Elimination Rate, kₑₗᵢₘ min⁻¹, or k_{FP elim} min⁻¹, may be expressed as the first phase of elimination of the intravenously administered 13C-cholate, calculation from Ln/linear regression of [13C-cholate] versus time (using only the 5- and 20-minute time points). Intravenously administered 13C-cholate is rapidly delivered to the liver via the hepatic artery. In contrast, the same 13C-cholate slowly transits to the liver via the portal vein due to the capacitance of the splanchnic vascular bed. Thus, the first phase of cholate elimination is more dependent upon clearance from the hepatic artery than from portal vein. The Volume of distribution, V_{d}, L kg⁻¹: The body's volume into which cholate is distributed. This is calculated from the intercept on the Y axis of the Ln/linear regression of [13C-cholate] versus time (using only the 5- and 20-min time points).

For example, a HepQuant® cholate liver function test (HepQuant, LLC) can be used to determine the presence and severity of liver diseases. A HepQuant® test can be a HepQuant® DSI test, HepQuant® SHUNT test or a HepQuant® STAT test. As described herein, The HepQuant® SHUNT test uses simultaneous orally administered d4-cholate and intravenously administered 13C-cholate (i.e. intravenous (24-¹³C cholate) and oral (2,2,4,4-²H cholate) together with mass spectrometry analysis of five serum samples from the subject to accurately measure liver function and generate a cholate SHUNT value or a disease severity index (DSI) value.

Test Outputs. Cholate concentrations (endogenous unlabeled CA, 13C-CA, and d4-CA) may be measured from the timed serum samples (collected 0, 5, 20, 45, 60, and 90 minutes after oral and i.v. coadministration) and concentrations of each labeled cholate as a function of time may be modeled as a spline curve in order to calculate the area under curve (AUC). The cholate SHUNT test parameters may include: DSI, indexed Hepatic Reserve, algebraic Hepatic Reserve, RISK-ACE, SHUNT %, RCA20, Systemic HFR, Portal HFR, cholate elimination rate, and volume of distribution.

The term “SHUNT test” refers to a previously disclosed QLFT (quantitative liver function test) used as a comprehensive assessment of hepatic blood flow and liver function. The SHUNT test may be used to determine plasma clearance of orally and intravenously administered distinguishable cholic acids in subjects with and without chronic liver disease. SHUNT fraction or percent quantifies the spillover of the PO d4-cholate into the systemic circulation from the ratio of the clearance of the intravenously administered 13C-cholate to the clearance of the orally administered d4-cholate. In the SHUNT test, at least 5 blood samples are analyzed which have been drawn from a patient at intervals over a period of about 90 minutes after oral and intravenous administration of differentiable cholates. The SHUNT test is disclosed in Everson et al., U.S. Pat. Nos. 8,613,904, 8,613,904, 9,639,665, 8,778,299, 9,417,230, and 10,215,746, each of which is incorporated herein by reference in its entirety. These studies demonstrated reduced clearance of cholate in patients who had either hepatocellular damage or portosystemic shunting. The “SHUNT test value” refers to a number (in %). The term “SHUNT %” represents a quantitative measurement of portal-systemic shunting. SHUNT % is a measurement of the percentage of spillover of the orally administered d4-cholate. The first-pass hepatic elimination of cholate in percent of orally administered cholate is defined as (100%—SHUNT). Analysis of samples for stable isotopically labeled cholates is performed by, e.g., GC-MS, following sample derivitization, or LC-MS, without sample derivitization, or LC-MS/MS, or MS/MS. The ratio of the AUCs of orally to intravenously administered cholic acid, corrected for administered doses, defines cholate shunt. The cholate shunt can be calculated using the formula: AUCₒᵣₐₗ/AUCᵢᵥ×Doseᵢᵥ/Doseₒᵣₐₗ×100%, wherein AUCₒᵣₐₗ is the area under the curve of the serum concentrations of the orally adminstered cholic acid and AUCᵢᵥ is the area under the curve of the intravenously administered cholic acid. The SHUNT test allows measurement of first-pass hepatic elimination of bile acids from the portal circulation.

The current HepQuant cholate SHUNT test uses two forms of cholate, d4- and 13C-cholates (stable nonradioactive isotopes), as liver-specific molecular probes of hepatocyte uptake. The test parameters include:

(i) Clearance of orally administered d4-cholate from the portal circulation (Portal Hepatic Filtration Rate (Portal HFR)),

(ii) Clearance of intravenously administered 13C-cholate from the systemic circulation (Systemic HFR),

(iii) Spillover of the orally-administered d4-cholate into the systemic compartment (i.e., portal-systemic shunting or SHUNT),

(iii) A Disease Severity Index (DSI),

(iv) A DSI-derived measure of Hepatic Reserve (HR), and

(v) STAT, the d4-cholate concentration in the 60 minute blood sample. STAT correlates well with DSI and can be used to estimate DSI.

The SHUNT fraction or percent quantifies the spillover of the PO d4-cholate into the systemic circulation from the ratio of the clearance of the intravenously administered 13C-cholate to the clearance of the orally administered d4-cholate.

DSI is an index, or score, that encompasses the cholate clearances from both systemic and portal circulations. DSI has a range from 0 (healthy) to 50 (severe end-stage disease) and is calculated from both HFRs.

The term “Cholate Elimination Rate”, kₑₗᵢₘ min⁻¹ represents the first phase of elimination of the intravenously administered 13C-cholate, calculation from Ln/linear regression of [13C-cholate] versus time (using only the 5- and 20-minute time points). Intravenously administered 13C-cholate is rapidly delivered to the liver via the hepatic artery. In contrast, the same 13C-cholate slowly transits to the liver via the portal vein due to the capacitance of the splanchnic vascular bed. Thus, the first phase of cholate elimination is more dependent upon clearance from the hepatic artery than from portal vein.

The term “Volume of distribution”, V_{d}, L kg⁻¹ represents the body's volume into which cholate is distributed. This is calculated from the intercept on the Y axis of the Ln/linear regression of [13C-cholate] versus time (using only the 5- and 20-min time points).

The acronym “IV” or “iv” refers to intravenous route of administration.

The acronym “PO” refers to per oral route of administration.

The acronym “PHM” refers to perfused hepatic mass.

The acronym “SF” refers to shunt fraction, for example, as in liver SF, or cholate SF.

The term “oral cholate clearance” (Clₒᵣₐₗ) refers to clearance from the body of a subject of an orally administered cholate compound as measured by a blood or serum sample from the subject. Oral cholate clearance is used as a measure of portal blood flow. Orally administered cholic acid is absorbed across the epithelial lining cells of the small intestine, bound to albumin in the portal blood, and transported to the liver via the portal vein. Approximately 80% of cholic acid is extracted from the portal blood in its first pass through the liver. Cholic acid that escapes hepatic extraction exits the liver via hepatic veins that drain into the vena cava back to the heart, and is delivered to the systemic circulation. The area under the curve (AUC) of peripheral venous concentration versus time after oral administration of cholic acid quantifies the fraction of cholic acid escaping hepatic extraction and defines “oral cholate clearance”.

The term “portal hepatic filtration rate”, “portal HFR”, “FLOW test” refers to oral cholate clearance (portal hepatic filtration rate; portal HFR) used as a measure of portal blood flow, or portal circulation, obtained from analysis of concentration of distinguishable cholate compound in at least 5 blood samples drawn from a subject over a period of, for example, about 90 minutes after oral administration of a distinguishable cholate compound, for example, a distinguishable cholate. The units of portal HFR value are typically expressed as mL/min/kg, where kg refers to kg body weight of the subject. “Portal HFR”, mL min⁻¹ kg⁻¹ may be used to Model independent apparent clearance of orally administered d4-cholate, adjusted for body weight, and calculated from dose/AUC. FLOW test methods are disclosed in U.S. Pat. Nos. 8,778,299, 9,417,230, and 10,215,746, each of which is incorporated herein by reference in its entirety. “Systemic HFR”, mL min⁻¹ kg⁻¹ may be used to Model independent clearance of intravenously injected 13C-cholate, adjusted for body weight, and calculated from dose/AUC.

The term “intravenous cholate clearance” (Clᵢᵥ) refers to clearance of an intravenously administered cholate compound. Intravenously administered cholic acid, bound to albumin, distributes systemically and is delivered to the liver via both portal venous and hepatic arterial blood flow. The AUC of peripheral venous concentration versus time after intravenous administration of cholic acid is equivalent to 100% systemic delivery of cholic acid. The ratio of the AUCs of orally to intravenously administered cholic acid, corrected for administered doses, defines cholate shunt.

HepQuant®-STAT is a simpler test that uses only orally administered d4-cholate and a single serum sample from the subject to estimate liver function (STAT). HepQuant® SHUNT, HepQuant® DSI, and HepQuant® STAT have each been described in one or more of U.S. Pat. Nos. 8,613,904, 8,778,299, 8,961,925, 9,091,701, 9,417,230, 9,759,731, 10,215,746, 10,222,366, 10,520,517, and U.S. Ser. No. 17/227,042, each of which is incorporated herein by reference in its entirety. The term “STAT test” refers to an estimate of portal blood flow by analysis from one patient blood sample drawn at a defined period of time following oral administration of a differentiable cholate. In one aspect, the STAT test refers to analysis of a single blood sample drawn at a specific time point after oral administration of a differentiable cholate. In one specific aspect, the STAT test is a simplified convenient test intended for screening purposes that can reasonably estimate the portal blood flow (estimated flow rate) from a single blood sample taken 60 minutes after orally administered deuterated-cholate. In some embodiments, STAT, is the d4-cholate concentration in the 60 minute blood sample. STAT correlates well with DSI and can be used to estimate DSI. The STAT test value is typically expressed as a concentration, for example, micromolar (uM) concentration. STAT test methods are disclosed in U.S. Pat. Nos. 8,961,925, 10,222,366, each of which is incorporated herein by reference in its entirety. A STAT test value may be used to estimate portal HFR, as provided in U.S. Pat. Nos. 8,961,925, 10,222,366.

The term “DSI test” refers to a cholate Disease Severity Index test which is derived from one or more liver function test results based on hepatic blood flow. The DSI score is a function of the sum of cholate clearances from systemic and portal circulations adjusted to disease severity ranging from healthy subjects to end stage liver disease. DSI is a score without units representing a quantitative measurement of liver function. A disease severity index (DSI) value may be obtained in a patient by a method comprising (a) obtaining one or more liver function test values in a patient having or at risk of a chronic liver disease, wherein the one or more liver function test values are obtained from one or more liver function tests selected from the group consisting of SHUNT, portal hepatic filtration rate (portal HFR), and systemic hepatic filtration rate (systemic HFR); and (b) employing a disease severity index equation (DSI equation) to obtain a DSI value in the patient, wherein the DSI equation comprises one or more terms and a constant to obtain the DSI value, wherein at least one term of the DSI equation independently represents a liver function test value in the patient, or a mathematically transformed liver function test value in the patient from step; and the at least one term of the DSI equation is multiplied by a coefficient specific to the liver function test. DSI is an index, or score, that encompasses the cholate clearances from both systemic and portal circulations. DSI has a range from 0 (healthy) to 50 (severe end-stage disease) and is calculated from both HFRs. Based on the reproducibility of DSI, the minimum detectable difference indicating a change in liver function in a subject may be about 1.5 points, about 2 points, or about 3 points. DSI test methods and equations are disclosed in U.S. Pat. Nos. 9,091,701, 9,759,731, 10,520,517, each of which is incorporated herein by reference in its entirety. Additional DSI equations have been developed and are provided herein.

DSI is a function of Shunt and Portal HFR and Systemic HFR, so DSI may be determined using a Generic DSI equation 1:

DSI=f(Shunt, Portal HFR, Systemic HFR)

Specific DSI equations are provided in U.S. Pat. Nos. 9,091,701, 9,759,731, 10,520,517, each of which is incorporated herein by reference in its entirety.

Additional specific DSI equations include the following example equations.

DSI equation 2 employs SHUNT, portal HFR and systemic HFR patient values.

DSI Equation 2:

DSI=A (Shunt)+B (Log Portal HFR)+C (log Systemic HFR)+D, wherein the constants A, B, C, D for use in DSI Equation 2 are shown in Table 10.

TABLE 10
Constants and coefficients for use in DSI Equation 2

| | A | B | C | D |
|---|---|---|---|---|
| DSI 3.1 | 5.75 | 7.22 | 8.45 | 50 |
| DSI 3.2 | 5.75 | 7.22 | 9.28 | 51.27 |
| DSI 3.3 | 5.34 | 6.65 | 8.57 | 44.66 |
| DSI 3.4 | 5.3 | 6.6 | 8.7 | 44.7 |

DSI equation 3 employs portal HFR and systemic HFR patient values.

$$DSI = A \sqrt{{( B - Log Portal HFR )}^{2} + {( C - Log Systemic HFR )}^{2}} \qquad DSI Equation 3$$

Constants and coefficients for use in Equation 3 are shown in Table 11.

TABLE 11
Constants and Coefficients for use in DSI Equation 3

| | A | B | C |
|---|---|---|---|
| DSI rtuln | 10.86186 | 3.94527 | 2.37168 |

In some embodiments, a SHUNT test value in the patient may be used in the DSI equation, and the SHUNT test value is determined by a method comprising receiving a plurality of blood or serum samples collected from the patient having PSC, following oral administration of a dose of a first distinguishable cholate (doseₒᵣₐₗ) to the patient and simultaneous intravenous co-administration of a dose of a second distinguishable cholate (doseᵢᵥ) to the patient, wherein the samples have been collected over intervals spanning a period of time after administration; quantifying the concentration of the first and the second distinguishable cholates in each sample; generating an individualized oral clearance curve from the concentration of the first distinguishable cholate in each sample comprising using a computer algorithm curve fitting to a model oral distinguishable cholate clearance curve and computing the area under the individualized oral clearance curve (AUCoral); generating an individualized intravenous clearance curve from the concentration of the second distinguishable cholate in each sample by use of a computer algorithm curve fitting to a model intravenous second distinguishable cholate clearance curve and computing the area under the individualized intravenous clearance curve (AUCiv); and calculating the shunt value in the patient using the formula:

AUCₒᵣₐₗ/AUCᵢᵥ×Doseᵢᵥ/Doseₒᵣₐₗ×100%.

The term “Hepatic Reserve” (HR) refers to percentage of maximum hepatic functional capacity measured by DSI, indexed hepatic reserve may be normalized to the DSI range in subjects of lean body mass.

HR (algebraic) is simply an algebraic conversion of the DSI value in the subject: HR=[100−(2×DSI)].

Indexed HR, is normalized against the results within a cohort of normal lean controls.

One formula for Hepatic Reserve (indexed) begins with the DSI equation:

$$DSI = A \sqrt{{( ln ( \frac{b}{{HFR}_{F}} ) )}^{2} + {( ln ( \frac{c}{{HFR}_{z}} ) )}^{2}} . \qquad Eqn$$

But, in contrast to DSI, a minimal limit is placed on the range of normal—the mean values for Portal HFR and Systemic HFR minus one SD of the mean for each in healthy controls of lean body mass (y and z, respectively). Thus, the HRindexed formula may be written as:

$$HRindexed = HR = 100 - ( X \sqrt{{( ln ( \frac{y}{{HFR}_{F}} ) )}^{2} + {( ln ( \frac{z}{{HFR}_{z}} ) )}^{2}} ) , \qquad Eqn .$$

wherein:
X is a scaling multiplier from 20 to 35 (optionally 29.40578) to yield a range from 100 (normal hepatic reserve) to 0 (no hepatic reserve),
y is the minimum value for Portal HFR determined from the mean values for Portal HFR minus one SD of the mean for each in a plurality of healthy controls of lean body mass in a range or 15-40, optionally wherein y=29.1; and
z is the minimum value for Systemic HFR determined from the mean values for Systemic HFR minus one SD of the mean for each in the plurality of healthy controls of lean body mass in a range of 4-10, optionally wherein z=6.52. The variables in the HR equation, y, z, HFRₚ, and HFRs, are all clearance values with units of mL min⁻¹ kg⁻¹—but, the units drop in the equation due to factoring the variables as ratios.

Based on the range of HFRs in 30 lean controls (Portal HFR 29.10±9.04 mL min⁻¹ kg⁻¹; Systemic HFR 6.52±1.49 mL min⁻¹ kg⁻¹), the minimum values for normal Portal HFR and Systemic HFR were set at:

| | | |
|---|---|---|
| Portal HFR | y = 29.1 | (range 20-40, or 25-35) |
| Systemic HFR | z = 6.52 | (range 4-12, or 5-10). |

Use of range in lean controls, versus all controls, allows detection of changes in HR in overweight and obese subjects for possible underlying fatty liver disease. As HFRₚ and HFRₛ approach y and z, respectively, HR approaches 100—“NORMAL HEPATIC RESERVE”. As HFRs approach 1, HR approaches 0—“NO HEPATIC RESERVE”.

The term “RCA20” represents the amount of the intravenously administered cholate compound, such as ¹³C-CA, that remains in the circulation 20 minutes after the intravenous injection. The formula for RCA20 may be expressed as:

RCA20=(1−([¹³C CA]ₜ₌₀−[¹³C CA]ₜ₌₂₀)/[¹³C CA]ₜ₌₀)×100%, Eqn.:

wherein RCA20 represents the amount of the intravenously administered 13C-CA that remains in the circulation 20 minutes after the intravenous injection. [¹³C CA]ₜ₌₀ is determined from Ln/linear regression of [13C-CA] versus time. RCA20 can be compared to R15 for ICG data. The indocyanine green (ICG) clearance test (K) and retention rate at 15 minutes (R15) have been used as one indicator of liver function for example in patients with cirrhosis.

The term “Quantitative Liver Function Test” (QLFT), refers to assays that measure the liver's ability to metabolize or extract test compounds, can identify patients with impaired hepatic function at earlier stages of disease, and possibly define risk for cirrhosis, splenomegaly, and varices. One of these assays is the cholate shunt assay where the clearance of cholate is assessed by analyzing bodily fluid samples after exogenous cholate has been taken up by the body.

The DSI value can be used to determine the extent of liver function, and therefore the extent of liver damage/disease in the subject. Thus, the DSI value can be used to diagnose a subject with a disease. For example, a DSI greater than 16.5 can indicate that the subject will likely have biopsy-proven NASH. A DSI value can also be used to determine the percent likelihood of endoscopic finding of varices in subjects with NASH or hepatitis C virus infection (HCV). For example, a DSI value greater than 25 indicates a 50% probability of varices in subjects with NASH or HCV. A DSI value greater than 30 indicates a 75% probability of varices in a subject with NASH. A DSI value in a subject having a chronic liver disease may be used for prediction of future clinical outcomes, wherein a DSI>19 indicates an increased risk of clinical outcomes in the patient.

A healthy control subject may exhibit a SHUNT % of about 24% and a DSI value of about 10. A subject with advanced fibrosis (F3) may exhibit a SHUNT % of about 39% and a DSI value of 18. A subject with compensated cirrhosis (F4)(CP A) may exhibit a SHUNT % of 51% and a DSI value of 26. A subject with decompensated cirrhosis (F4)(CP B) may exhibit a SHUNT % of 83% and a DSI value of 35.

In some embodiments, the method for determining a portal HFR value in a subject, for example, having or suspected of having or at risk of a chronic liver disease, comprises

(i) receiving a plurality of blood or serum samples collected from a subject having or at risk of a chronic liver disease, following oral administration of a dose of a distinguishable cholate compound (doseₒᵣₐₗ) to the subject, wherein the samples have been collected from the subject over intervals spanning a period of time of less than 3 hours after administration;

(ii) measuring concentration of the distinguishable compound in each sample comprising a method selected from the group consisting of GC-MS, LC-MS, LC-MS/MS, and MS/MS;

(iii) generating an individualized oral clearance curve from the concentration of the distinguishable cholate compound in each sample comprising using a computer algorithm curve fitting to a model distinguishable compound clearance curve;

(iv) computing the area under the individualized oral clearance curve (AUC)(mg/mL/min) and dividing the dose (in mg) by AUC of the orally administered distinguishable compound to obtain the oral distinguishable compound clearance in the subject; and

(v) dividing the oral distinguishable compound clearance by the weight of the subject in kg to obtain the portal HFR value in the subject (mL/min/kg).

In some embodiments, the method for determining a systemic HFR value in a subject, for example, having or suspected of having or at risk of a chronic liver disease, comprises:

(i) receiving a plurality of blood or serum samples collected from a subject having or at risk of a chronic liver disease, following intravenous administration of a dose of a distinguishable cholate compound (doseᵢᵥ) to the subject, wherein the samples have been collected from the subject over intervals spanning a period of time of less than 3 hours after administration;

(ii) measuring concentration of the distinguishable cholate compound in each sample comprising a method selected from the group consisting of GC-MS, LC-MS, LC-MS/MS, and MS/MS;

(iii) generating an individualized intravenous clearance curve from the concentration of the distinguishable cholate compound in each sample comprising using a computer algorithm curve fitting to a model distinguishable compound clearance curve;

(iv) computing the area under the individualized intravenous clearance curve (AUC)(mg/mL/min) and dividing the dose (in mg) by AUC of the intravenously administered distinguishable compound to obtain the intravenous distinguishable compound clearance in the subject; and

(v) dividing the intravenous distinguishable cholate compound clearance by the weight of the subject in kg to obtain the systemic HFR value in the subject (mL/min/kg).

The distinguishable cholate compound may be is an isotopically cholate compound, preferably a stable isotope labeled cholate compound. For example, the stable isotope labeled cholate compound may be a cholic acid-2,2,4,4-D4 (D4-CA; CA-D4), 24-¹³C-cholic acid (¹³C-CA), 2,2,3,4,4-d₅ cholic acid (D₅-CA), or 3,6,6,7,8,11,11,12-d8 cholic acid (D8-CA).

The liquid chromatography (LC) may be any appropriate liquid chromatography. The LC, for example, may be selected from the group consisting high performance LC (HPLC) and ultra-high performance LC (UPLC).

The mass spectrometer (MS) typically comprises an ion source system and a mass resolution/detection system. The ion source system may be selected from the group consisting of electrospray ionization (ES), matrix-assisted laser desorption/ionization (MALDI), fast atom bombardment (FAB), chemical ionization (CI), atmospheric pressure chemical ionization (APCI), liquid secondary ionization (LSI), laser diode thermal desorption (LDTD), and surface-enhanced laser desorption/ionization (SELDI). The mass resolution/detection system may be selected from the group consisting of triple quadrupole mass spectrometer (MS/MS); single quadrupole mass spectrometer (MS); Fourier-transform mass spectrometer (FT-MS); and time-of-flight mass spectrometer (TOF-MS).

In some embodiments, the method for determining a hepatic cholate shunt (SHUNT %) value and/or relative hepatic function in a subject, for example, having or suspected of having or at risk of a hepatic disorder or chronic liver disease, comprises
(a) obtaining a multiplicity of blood or serum samples collected from a subject over intervals for a period of less than 3 hours after the subject had been orally administered a first distinguishable cholate compound and simultaneously intravenously administered a second distinguishable cholate compound;
(b) quantifying the first and the second distinguishable compounds in the samples comprising a method selected from the group consisting of GC-MS, LC-MS, LC-MS/MS;
(c) calculating the hepatic shunt in the subject using the formula:

AUCoral/AUCiv×Doseiv/Doseoral×100%;
wherein AUCoral is the area under the curve of the serum concentrations of the first distinguishable cholate compound and AUCiv is the area under the curve of the second distinguishable cholate compound to obtain the SHUNT % value in the subject.

The SHUNT % method may further comprise comparing the SHUNT % value in the subject to a SHUNT % cutoff value or cutoffs of values established from a known subject population wherein the hepatic shunt in the subject compared to shunt cutoff value or cutoffs of values is an indicator of relative hepatic function of the subject.

The blood or serum samples may be collected from the subject at 2 or more, 3 or more, 3 or more, 4 or more, 5 or more, 6 or more, 7 or more, 8 or more, 9 or more, 10 or more, 11 or more, 12 or more, 13 or more or 15 or more time points, preferably collected over intervals spanning a period of time within about 3 hrs, or about 90 minutes or less after administration. The samples may comprise 5 samples collected within about 90 minutes after administration. The samples may be collected at about 5, 20, 45, 60 and 90 minutes after the administration of the distinguishable cholate compounds.

In some embodiments, the method for determining a disease severity index (DSI) value in a subject comprises
(a) obtaining one or more liver function test values in a subject having or at risk of a chronic liver disease, wherein the one or more liver function test values are obtained from one or more liver function tests selected from the group consisting of SHUNT, portal hepatic filtration rate (portal HFR), and systemic hepatic filtration rate (systemic HFR), wherein the liver function tests comprise measuring a distinguishable compound in a blood or serum sample comprising the method according to any one of claims 1 to 29; and
(b) employing a disease severity index equation (DSI equation) to obtain a DSI value in the subject, wherein the DSI equation comprises one or more terms and a constant to obtain the DSI value, wherein
   at least one term of the DSI equation independently represents a liver function test value in the subject from step (a) or a mathematically transformed liver function test value in the subject from step (a); and
   the at least one term of the DSI equation is multiplied by a coefficient specific to the liver function test.

In some embodiments, the DSI equation may be selected from the group consisting of:

(I) DSI=f(Shunt, Portal HFR, Systemic HFR), wherein Shunt is a shunt value in the subject, portal HFR is a portal HFR value in the subject, and systemic HFR is an systemic HFR value in the subject;

(II) DSI=A (Shunt)+B (Log Portal HFR)+C (log Systemic HFR)+D, wherein A=a number from 5 to 6; B=a number from 6 to 8; C=a number from 8 to 10; D=a number from 40 to 60;

(III)

DSI=A√(B−Log Portal HFR)²+(C−Log Systemic HFR)²,

wherein A=a number from 8 to 12; B=a number from 3 to 5; C=a number from 1.5 to 3.5;

$$DSI = A \sqrt{{( B - ( ln {HFR}_{F} ) )}^{2} + {( C - ( ln HFR \text{?} ) )}^{2}} ; \qquad ( IV )$$

$$\text{?} \text{indicates text missing or illegible when filed}$$

wherein A=a scaling multiplier from 8 to 12 (optionally 10.86) to yield a range from 0 (no disease) to 50 (end-stage disease), B is the natural logarithm of the maximum value for Portal HFR, b, and C is the natural logarithm of the maximum value for Systemic HFR, c; and

$$DSI = A \sqrt{{( ln ( \frac{b}{{HFR}_{F}} ) )}^{2} + {( ln ( \frac{c}{HFR \text{?}} ) )}^{2}} , \qquad ( V )$$

$$\text{?} \text{indicates text missing or illegible when filed}$$

wherein A=a scaling multiplier from 8 to 12 (optionally 10.86) to yield a range from 0 (no disease) to 50 (end-stage disease), b is the maximum value for Portal HFR in a range from 25-75, and c is the maximum value for Systemic HFR in a range from 5-15.

The DSI method may further comprise comparing the DSI value in the subject to one or more DSI cut-off values, one or more normal healthy controls, or one or more DSI values within the subject over time.

The comparing of the DSI value within the subject over time may be used to monitor the effectiveness of a treatment of chronic liver disease in the subject, wherein a decrease in the DSI value within the subject over time is indicative of treatment effectiveness, optionally wherein the decrease in DSI value within the subject over time is at least about −1.5 points, at least about—2 points, or at least about—3 points.

The comparing of the DSI value in the subject to one or more DSI cut-off values may be indicative of at least one clinical outcome. For example, the clinical outcome may be selected from the group consisting of Child-Turcotte-Pugh (CTP) increase, varices, encephalopathy, ascites, and liver related death.

The comparing of the DSI value in the subject over time may be used to monitor the need for treatment of chronic liver disease in the subject, wherein an increase in the DSI value within the subject over time is indicative of a need for treatment in the subject.

In some embodiments, the method for determining a cholate STAT value in a subject comprises

obtaining a blood or serum sample from a subject having or suspected of having or at risk of a chronic liver disease, following oral administration of a distinguishable cholate compound to the subject, wherein the blood or serum sample was collected from the subject less than 3 hours after oral administration of the distinguishable compound to the subject, optionally wherein the blood or serum sample consists of a single blood or serum sample;

measuring the concentration of the orally administered distinguishable compound in the blood or serum sample from the subject, comprising quantifying the concentration of the distinguishable cholate compound in the sample comprising a method selected from the group consisting of GC-MS, LC-MS, LC-MS/MS to obtain a STAT value in the subject.

The method for determining a cholate STAT value in a subject may further comprise

comparing the STAT value in the subject to (i) a distinguishable cholate compound STAT cutoff value or cutoffs of values established from a known subject population, and/or to (ii) a STAT reference value from the same subject obtained from one or more earlier blood or serum samples from the subject over time.

The comparing of the STAT value in the subject over time may be used to monitor the effectiveness of a treatment of chronic liver disease in the subject, wherein a decrease in concentration of the distinguishable compound over time is indicative of treatment effectiveness.

The blood or serum samples may be collected at one or more time points after the oral administration of the distinguishable compound, selected from the group consisting of baseline, 5, 10, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, and 180 minutes, or any time point in between; optionally wherein the blood or serum sample is a single blood or serum sample collected at a single specific time point. The single blood or serum sample may be collected at one time point selected from about 45, about 60 or about 90 minutes after oral administration of the distinguishable cholate compound.

The concentration of distinguishable cholate compound in the single blood or serum sample (STAT value) may be used in a method to estimate portal hepatic filtration rate (portal HFR) (FLOW) in the subject.

The method for estimation of portal HFR (FLOW) in the subject further may comprise

converting the concentration of the distinguishable compound in the single blood or serum sample (STAT value) by using an equation into an estimated portal HFR (FLOW) (mL/min/kg) in the subject; and

comparing the estimated portal HFR in the subject to a portal HFR (FLOW) cutoff value or cutoffs of values established from a known subject population or within the subject over time.

The comparing of the estimated portal HFR (FLOW) (mL/min/kg) values in the subject over time may be used to monitor the effectiveness of a treatment of chronic liver disease in the subject, wherein an increase in estimated portal HFR (FLOW) (mL/min/kg) value over time is indicative of treatment effectiveness.

The equation for converting the STAT value into an estimated portal HFR (FLOW)(mL/min/kg) value in the subject may be:

y=A(x)+C, wherein
x=LOG estimated portal HFR (FLOW) value (mL/min/kg) in the subject;
y=LOG STAT value (μM adjusted to kg bodyweight) in the subject;
A=slope coefficient from 0.9 to 1.1; and
C=a constant from −0.05 to 0.05.
   In some embodiments, the cutoffs for FLOW (portal HFR) and SHUNT test results may be FLOW cutoff values (5, 10 and 20 mL/min/kg for marked severe, moderate, and mild chronic liver disease, respectively) and SHUNT cutoff values (26%, 43%, and 60% for mild, moderate and marked severe chronic disease, respectively). The cutoffs may be developed from healthy controls, and one or more disease cohorts, e.g., PSC patients, and HCV patients.
   A known population of patients may be used to establish various cutoff values for the STAT, single-point screening test at a particular selected time point for drawing the single blood sample following oral administration of the distinguishable cholate.

The STAT value for an individual patient may be compared to an established cutoff values. For example, the STAT test may be used in a patient suspected of having liver disease. A STAT test result from a patient falling within the range of about 0 to about 0.6 uM (“A” range) is likely to be predictive that the FLOW test result will also fall within the normal range for portal circulation. The patient with a STAT test result falling within the A range can be followed, for example, by use of an annual STAT test. A STAT test result falling within the range of about 0.6 uM to about 1.50 uM (“B” range) is likely to be predictive that the FLOW test result will fall within a compromised range for portal circulation. The patient with a STAT test result falling within the B range should be further evaluated, for example, with the FLOW, SHUNT and/or tests, for assessment of portal circulation and cholate clearances and shunt, respectively. A STAT test result falling above about 1.50 uM (“C” range) is likely to be predictive of advanced disease. A patient with a STAT test result falling within the C range should be further evaluated, for example, by SHUNT, DSI, EGD (upper endoscopy, esophagogastroduodenoscopy) and/or HCC (hepatocellular carcinoma) screening.

In some embodiments, a DSI value below cutoff <DSI 14 may be indicative of a healthy subject. In some embodiments, in a patient having a chronic liver disease, a DSI value between 15 and 25 may be indicative of mild disease. In some embodiments, in a patient having a chronic liver disease, a DSI value between 25 and 35 may be indicative of moderate disease. In some embodiments, in a patient having a chronic liver disease, a DSI value between >35 may be indicative of severe disease. In some embodiments, in a patient having a chronic liver disease, a DSI a cutoff >18 may be indicative of portal hypertension (PHTN). In some embodiments, in a patient having a chronic liver disease, a DSI value >18 may be indicative of esophageal varices. In some embodiments, in a patient having a chronic liver disease, a DSI value >19 may be indicative of medium to large esophageal varices. In some embodiments, in a patient having a chronic liver disease, a DSI value >19 may be indicative of a future clinical outcome. In some embodiments, in a patient having a chronic liver disease, a DSI value >23, may be predictive of a future clinical outcome. In some embodiments, in a patient having a chronic liver disease, a DSI value >25 may be indicative of cirrhosis. In some embodiments, in a patient having a chronic liver disease, a DSI value >36 may be indicative of decompensation.

Chronic Liver Diseases

The disclosure provides methods and compositions of treating a chronic liver disease or disorder. The chronic liver disease or disorder may be selected from the group consisting of non-alcoholic steatohepatitis (NASH), non-alcoholic fatty liver disease (NAFLD), alcoholic liver disease, drug-induced liver disease, chronic hepatitis C, chronic hepatitis B, cytomegalovirus, Epstein Barr virus, portal hypertension, cryptogenic cirrhosis, alpha 1-antitrypsin disease, hemochromatosis, nodular regenerative hyperplasia, idiopathic liver disease, congenital liver diseases, haemochromatosis, Wilson's disease, autoimmune chronic hepatitis, primary biliary cirrhosis, primary sclerosing cholangitis (PSC), liver damage due to progressive fibrosis, or liver fibrosis, and hepatocellular carcinoma (HCC).

The term “Chronic Hepatitis C” (CHC) refers to a chronic liver disease caused by viral infection and resulting in liver inflammation, damage to the liver and cirrhosis. Hepatitis C is an infection caused by a blood-borne virus that attacks the liver and leads to inflammation. Many people infected with hepatitis C virus (HCV) do not exhibit symptoms until liver damage appears, sometimes years later, during routine medical tests.

The term “Alcoholic SteatoHepatitis” (ASH) refers to a chronic condition of inflammation of the liver which is caused by excessive drinking. Progressive inflammatory liver injury is associated with long-term heavy intake of ethanol and may progress to cirrhosis.

The term “Non-Alcoholic SteatoHepatitis” (NASH) refers to a serious chronic condition of liver inflammation, progressive from the less serious simple fatty liver condition called steatosis. Simple steatosis (alcoholic fatty liver) is an early and reversible consequence of excessive alcohol consumption. In people that don't drink much alcohol, the cause of fatty liver disease is less clear, but may be associated with factors such as obesity, high blood sugar, insulin resistance, or high levels of blood triglycerides. In certain cases the fat accumulation can be associated with inflammation and scarring in the liver. This more serious form of the disease is termed non-alcoholic steatohepatitis (NASH). NASH is associated with a much higher risk of liver fibrosis and cirrhosis than NAFLD. Patients with NASH have increased risk for hepatocellular carcinoma. NAFLD may progress to NASH with fibrosis cirrhosis and hepatocellular carcinoma.

The term “Non-Alcoholic Fatty Liver Disease” (NAFLD) refers to a common chronic liver disease characterized in part by a fatty liver condition with associated risk factors of obesity, metabolic syndrome, and insulin resistance. Both NAFLD and NASH are often associated with obesity, diabetes mellitus and asymptomatic elevations of serum ALT and gamma-GT. Ultrasound monitoring can suggest the presence of a fatty infiltration of the liver; differentiation between NAFLD and NASH, typically requires a liver biopsy.

Treatments for NAFLD including NASH may include exercise, weight loss and avoiding hepatotoxins or any substance that may damage the liver. In some embodiments, therapies include administration of antioxidants, cytoprotective agents, antidiabetic agents, insulin-sensitizing agents (e.g. metformin), anti-hyperlipidemic agents, other chemical compounds, such as fibrates, thiazolidinediones (i.e., rosiglitazone or pioglitazone), biguanides, statins, cannabinoids, and other therapeutic compounds or molecules that target nuclear receptors, angiotensin receptors, cannabinoid receptors or HMG-CoA reductase.

The term “Primary Sclerosing Cholangitis” (PSC) refers to a chronic liver disease caused by progressive inflammation and scarring of the bile ducts of the liver. Scarring of the bile ducts can block the flow of bile, causing cholestasis. The inflammation can lead to liver cirrhosis, liver failure and liver cancer. Chronic biliary obstruction causes portal tract fibrosis and ultimately biliary cirrhosis and liver failure. The definitive treatment is liver transplantation. Indications for transplantation include recurrent bacterial cholangitis, jaundice refractory to medical and endoscopic treatment, decompensated cirrhosis and complications of portal hypertension (PHTN). PSC progresses through chronic inflammation, fibrosis/cirrhosis, altered portal circulaton, portal hypertension and portal-systemic shunting to varices-ascites and encephalopathy. Altered portal flow is an indication of clinical complications.

Association of Diabetes Mellitus with Chronic Liver Disease

There is a bidirectional relationship between diabetes mellitus (DM) and liver cirrhosis: hereditary type 2 DM is a risk factor for chronic liver disease (CLD). On the other hand, DM may occur as a complication of cirrhosis. This type of diabetes is known as hepatogenous diabetes (HD). Retrospective studies have shown that DM is associated with an increased risk of hepatic complications and death in patients with liver cirrhosis. DM may be associated with hepatic encephalopathy, portal hypertension and bleeding from esophageal varices in decompensated patients. In a cohort of individuals with liver infection by hepatitis B virus (HBV), those who developed de novo DM had higher risk of developing cirrhosis and hepatic complications. In patients with chronic hepatitis C, DM was an independent predictor of hepatic complications such as ascites, spontaneous bacterial peritonitis, renal dysfunction and hepatocellular cancer (HCC). Garcia-Compean et al., 2015, Annals of Hepatology, 14(6):780-788.

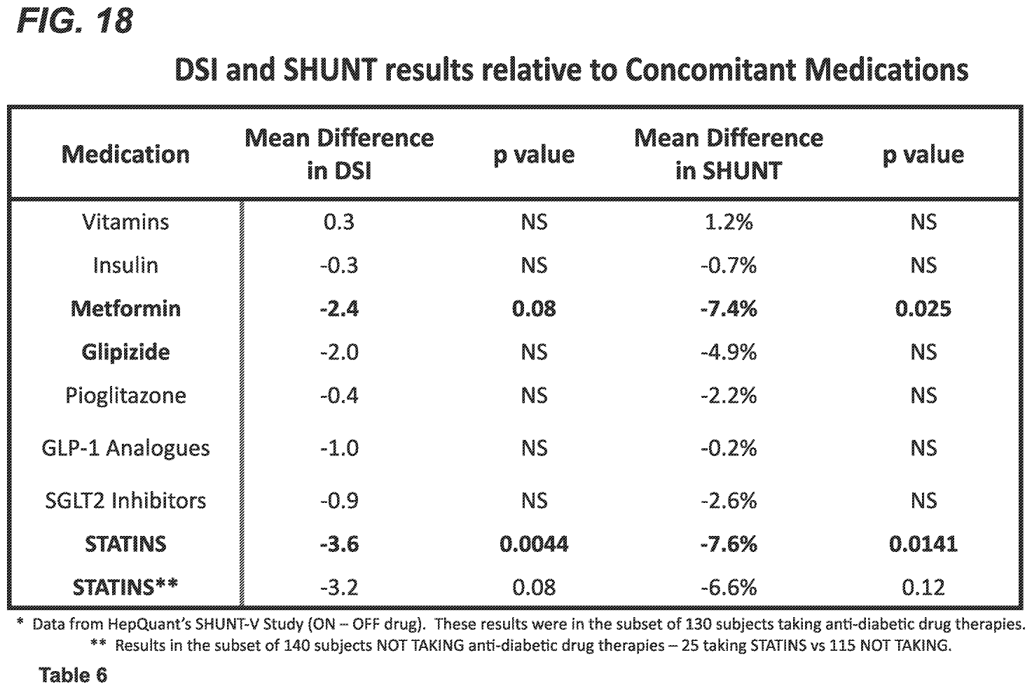

The disclosure provides methods of treating a chronic liver disease comprising coadministration of a statin and a biguanide. Optionally an additional agent selected from the group consisting of vitamin E and ursodeoxycholic acid may be coadministered. The chronic liver disease may be NASH. The chronic liver disease may be NAFLD. The chronic liver disease may be associated with diabetes mellitus. Surprisingly subjects taking a biguanide such as metformin and a statin exhibited improved liver function as measured in cholate function tests, including cholate SHUNT and DSI, as provided herein for example as shown in Table 6, FIG. 18.

Compositions

The disclosure provides methods of treating a chronic liver disease comprising administration of a statin and a biguanide. The disclosure provides compositions comprising a statin and a biguanide. The disclosure provides a fixed-dose composition comprising a statin and a biguanide.

A “statin” refers to a HMG-CoA (3-hydroxy-3-methylglutaryl-CoA) reductase inhibitors, which may be used in the prevention of hypercholesterolemia and related diseases. Statins are a competitive antagonists of HMG CoA, as they directly compete with the endogenous substrate for the active site cavity of HMGR. Statins are also noncompetitive with the cosubstrate NADPH (nicotinamide adenine dinucleotide phosphate). By blocking the HMGR enzyme they inhibit the synthesis of cholesterol via the mevalonate pathway. The end result is lower LDL (Low Density Lipoprotein), TG (Triglycerides) and total cholesterol levels as well as increased HDL (High Density Lipoprotein) levels in serum.

The compositions of the disclosure may comprise a statin. The statin may be selected from the group consisting of lovastatin, pravastatin, simvastatin, fluvastatin, cerivastatin, atorvastatin, pitavastatin, and rosuvastatin. The statins may include mevastatin, and cerivastatin. The methods of the disclosure may comprise use of a composition comprising a statin. In some embodiments, the statin is a type 1 statin or a type 2 statin. The statin may be a type 1 statin. Type 1 statins have a substituted decalin ring, similar to early statin mevastatin. The type 1 statin may be selected from the group consisting of lovastatin, pravastatin, and simvastatin. The statin may be a type 2 statin. Type 2 statins typically have a replacement of the butyryl group of type 1 statins with a fluorphenyl group of the type 2 statins. The type 2 statin may be selected from the group consisting of fluvastatin, cerivastatin, atorvastatin, and rosuvastatin. Certain statins are metabolized by cytochrome P450 enzymes. The statin may be a statin metabolized by cytochrome P450 enzyme CYP3A4 such as atorvastatin, lovastatin, or simvastatin. The statin may be a statin metabolized by cytochrome P450 enzyme CYP2C9 such as fluvastatin or rosuvastatin. The daily dose of the statin may be in a range from about 5 mg to about 100 mg daily, about 10 mg to about 80 mg daily, or about 20 mg to about 40 mg daily.

A “biguanide” refers to a class of drugs that function as antihyperglycemic agents, typically used for treatment of diabetes mellitus or prediabetes treatment. A biguanide is an organic compound comprising a substituted formula HN(C(NH)NH₂)₂.

The compositions of the disclosure may comprise a biguanide. The methods of the disclosure comprise use of a composition comprising biguanide. The biguanide may be metformin. Metformin is an asymmetric dimethylbiguanide. Other biguanides including phenformin and buformin have been withdrawn from the market in many countries due to side effects such as lactic acidosis. Still other biguanides have been used as antimalarial drugs including proguanil and chlorproguanil. The dose of biguanide may be in a range of from about 500 mg to about 2550 mg daily, about 800 to about 2,000 mg daily, or about 1,000 mg/daily to about 1,500 mg daily.

The disclosure provides a fixed-dose composition comprising a statin and a biguanide. The fixed dose combination may include from about 5 mg to about 100 mg daily of the statin and about 250 mg to about 2550 mg daily of the biguanide. The fixed dose combination may include from about 5 mg to about 100 mg daily of the statin and about 500 mg to about 2500 mg daily of the biguanide. The fixed dose combination may include from about 10 mg to about 80 mg daily of the statin and about 800 to about 2,000 mg daily of the biguanide.

The compositions of the disclosure may optionally further include vitamin E. The methods of the disclosure may comprise administration of a statin, a biguanide, and optionally vitamin E. The disclosure provides a fixed-dose composition comprising a statin and a biguanide, and optionally vitamin E.

Vitamin E is a group of eight fat soluble compounds that may include four tocopherols and four tocotrienols. Vitamin E deficiency, which is rare and usually due to an underlying problem with digesting dietary fat rather than from a diet low in vitamin E, can cause nerve problems. Vitamin E is a fat-soluble antioxidant protecting cell membranes from reactive oxygen species. Both the tocopherols and tocotrienols occur in a (alpha), β (beta), γ (gamma) and δ (delta) forms, as determined by the number and position of methyl groups on the chromanol ring. All eight of these vitamers feature a chromane double ring, with a hydroxyl group that can donate a hydrogen atom to reduce free radicals, and a hydrophobic side chain which allows for penetration into biological membranes. Of the many different forms of vitamin E, gamma-tocopherol (γ-tocopherol) is the most common form found in the North American diet, but alpha-tocopherol (α-tocopherol) may be the most biologically active. The vitamin E may contain an isomer selected from the group consisting of alpha-tocopherol, beta-tocopherol, gamma-tocopherol, delta-tocopherol, tocopheryl acetate, alpha-tocotrienol, beta-tocotrienol, gamma-tocotrienol, and delta-tocotrienol. The dose of vitamin E may be from about 50 IU to 1000 IU, 100 IU to 800 IU, or 200 IU to 400 IU daily.

The term “sulfonylurea” refers to a class of organic compounds used in medicine, for example as antidiabetic drugs which may be used in the management of diabetes mellitus type 2. Sulfonylureas act by increasing insulin release from the beta cells in the pancreas. Sulfonylureas bind to and close ATP-sensitive K⁺ (K_{ATP}) channels on the cell membrane of pancreatic beta cells, which depolarizes the cell by preventing potassium from exiting. This depolarization opens voltage-gated Ca²⁺ channels. The rise in intracellular calcium leads to increased fusion of insulin granulae with the cell membrane, and therefore increased secretion of mature insulin. Sulfonylureas may also sensitize 3-cells to glucose, that they limit glucose production in the liver, may decrease lipolysis (breakdown and release of fatty acids by adipose tissue) and may decrease clearance of insulin by the liver.

The compositions of the disclosure may comprise a statin and a sulfonylurea, and optionally a sulfonylurea. The methods of the disclosure may comprise administration of a composition comprising a statin and a biguanide, and optionally a sulfonylurea. The sulfonylurea may be selected from the group consisting of glipizide, tolazamide, chlorpropamide, glimepiride, glyburide, and glibenclamide. The daily dose of sulfonylurea may be in a range of from about 1.25 mg to about 20 mg/day, about 2.5 mg to about 10 mg/day, or about 5 to about 10 mg/day.

The compositions of the disclosure may optionally include a PPAR-gamma agonist such as a thiazolidinedione. The thiazolidinedione may be Pioglitazone, Rosiglitazone, or Lobeglitazone. For example, Pioglitazone may be used to lower blood glucose levels in type 2 diabetes either alone or in combination with a sulfonylurea, metformin, or insulin. Pioglitazone selectively stimulates the nuclear receptor peroxisome proliferator-activated receptor gamma (PPAR-γ) and to a lesser extent PPAR-α.

Peroxisome proliferator-activated receptor gamma (PPAR-γ or PPARG), also known as the glitazone receptor, or NR1C3 (nuclear receptor subfamily 1, group C, member 3) is a type II nuclear receptor (protein regulating genes). PPARG is mainly present in adipose tissue, colon and macrophages. Two isoforms of PPARG are detected in the human and in the mouse: PPAR-γ1 (found in nearly all tissues except muscle) and PPAR-γ2 (mostly found in adipose tissue and the intestine). PPARG regulates fatty acid storage and glucose metabolism. The genes activated by PPARG stimulate lipid uptake and adipogenesis by fat cells. PPAR-gamma may be implicated in the pathology of numerous diseases including obesity, diabetes, atherosclerosis, and cancer. PPAR-gamma agonists may be used in the treatment of hyperlipidaemia and hyperglycemia.

The daily dose of PPAR-gamma agonist such as piaglitazone may be in the range of from about 7.5 mg to about 45 mg, or from about 15 mg to about 30 mg. The compositions of the disclosure may optionally include ursodeoxycholic acid, (UDCA) which is a naturally occurring bile acid found in small quantities in normal human bile. It may be used, for example, in the treatment of primary biliary cirrhosis (PBC), or prevention or dissolution of gallstones. The daily dose of ursodeoxycholic acid may be in a range of from about 125 mg to about 1050 mg/day, or from about 300 mg to about 600 mg/day.

### EXAMPLES

### Example 1. Impact of NASH and Diabetes Mellitus on Clinical Scores (CP, MELD, Lab Results), Transient and Shear-Wave Elastography, Endoscopic Findings, and HepQuant Cholate SHUNT Test Results

A clinical study entitled “SHUNT-V” was performed to stratify risk for esophageal varices with a goal of identifying patients that require endoscopy to identify and treat varices. An analysis of the impact of NASH and diabetes mellitus on clinical scores (CP, MELD, lab results), transient and shear-wave elastography, endoscopic findings, and HepQuant cholate SHUNT test results was performed.

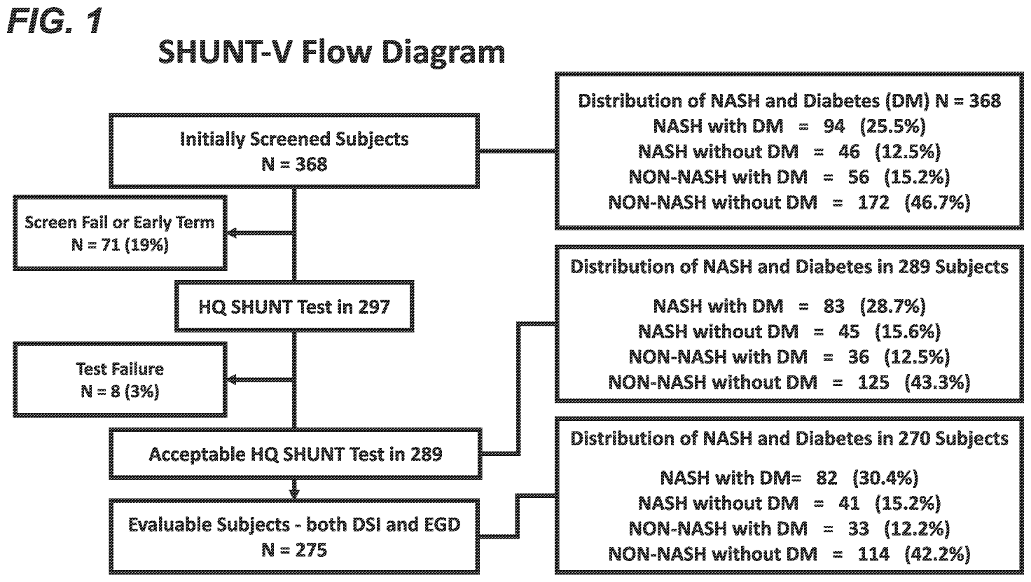

Initially screened subjects (n=368) exhibited a distribution of NASH and diabetes mellitus (DM) as shown in FIG. 1. HepQuant SHUNT test was performed in 297 subjects. Of the evaluable subjects (n=275), DSI and EGD was performed. Distribution of NASH and diabetes in 270 subjects showed the number of subjects with NASH with DM was 82 (30.4%), NASH without DM=41 (15.2%), Non-NASH with DM=33 (12.2%), and non-NASH without DM=114 (42.2%), as shown in FIG. 1.

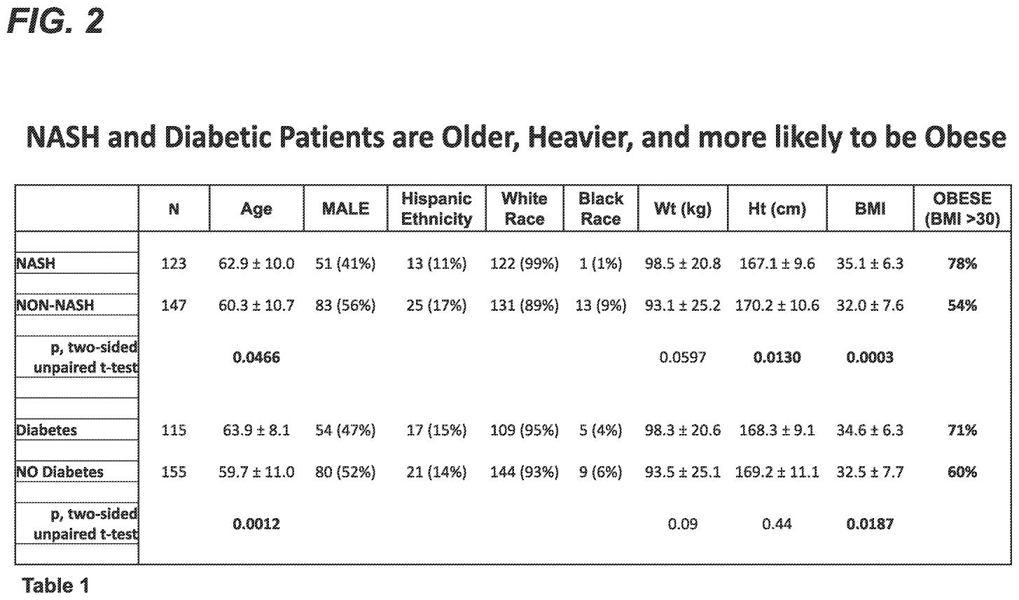

The 270 subjects were sorted for analysis as to NASH and non-NASH and DM and non-DM groups and mean age, sex, weight (kg), height (cm), body mass index (BMI), and rate of obesity (BMI>30) were determined, as shown in FIG. 2 The NASH and diabetic patients were found to be older, heavier, and more likely to be obese.

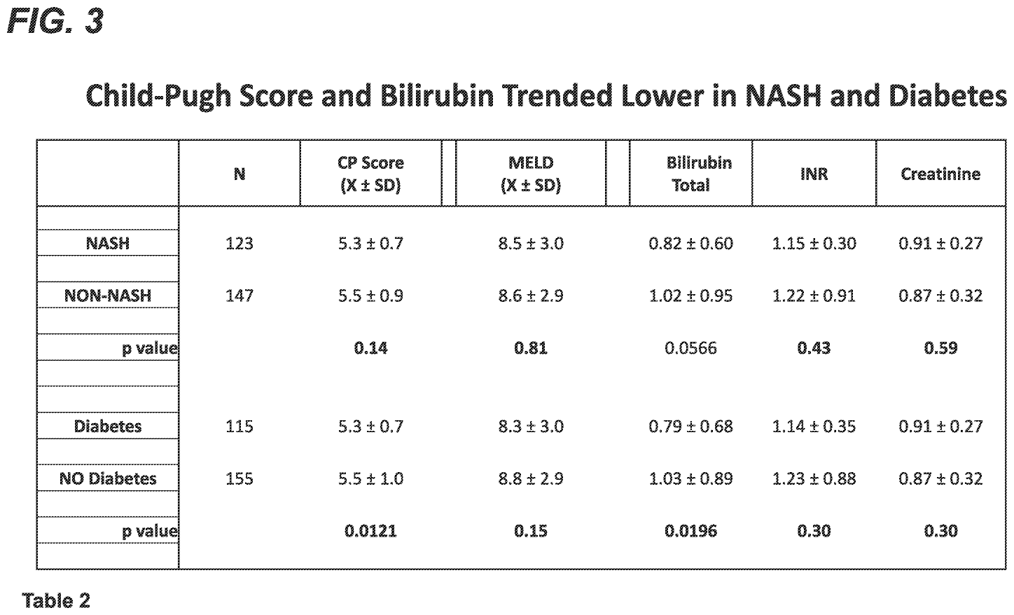

The 270 subjects were sorted for analysis as to NASH and non-NASH and DM and non-DM groups and mean Child-Pugh (CP) score (±SD), mean MELD score (±SD), mean total bilirubin (±SD), avg. INR (International Normalized Ratio prothrombin time) (±SD), and avg. creatinine (±SD) are shown in FIG. 3. Surprisingly, Child-Pugh (CP) score and bilirubin trended lower in NASH and diabetic patients.

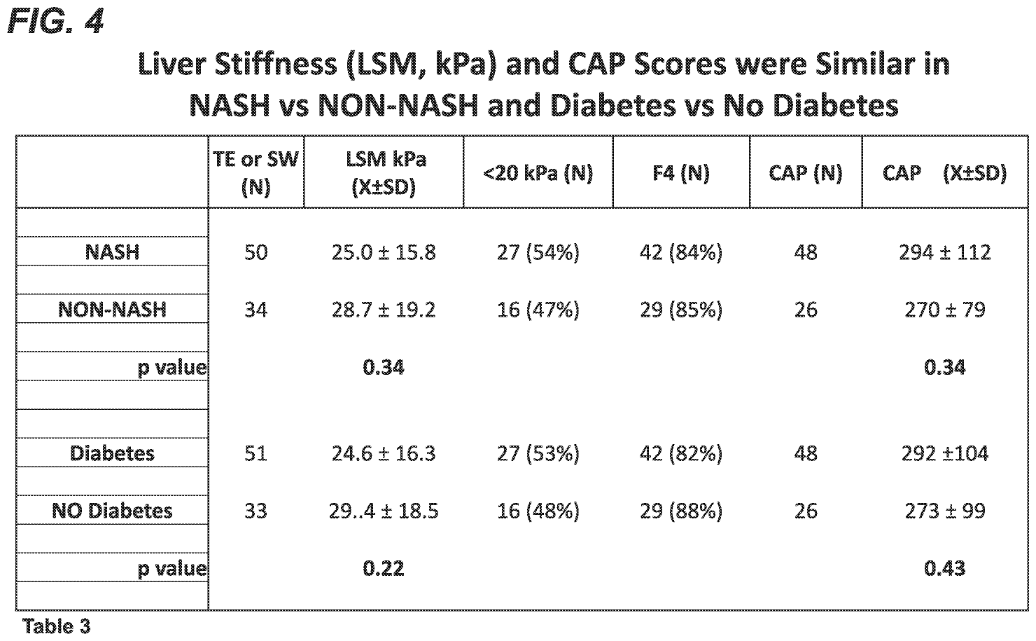

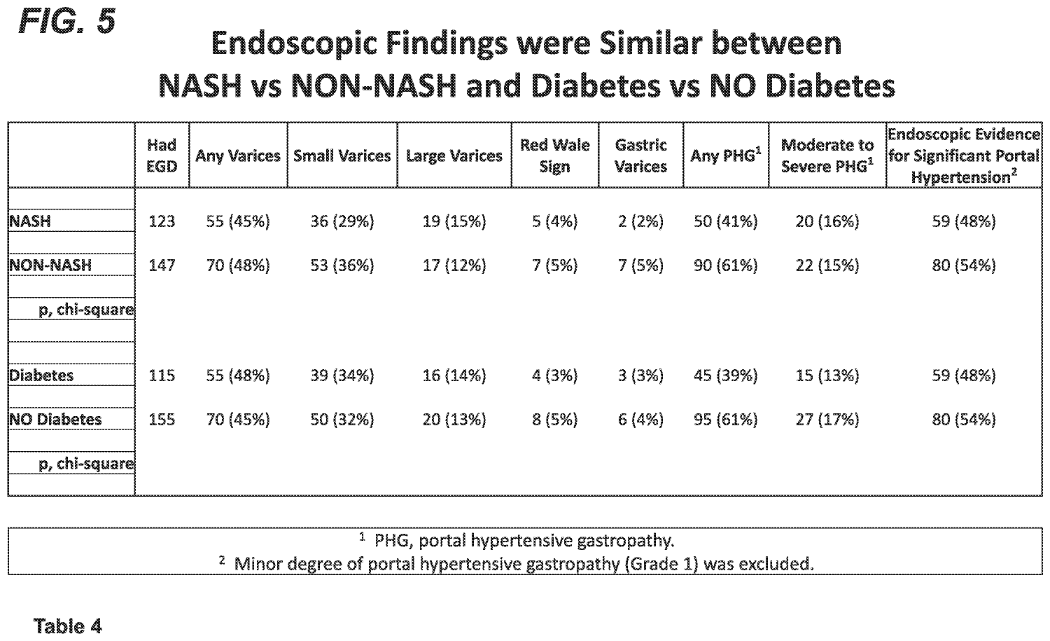

Liver stiffness measure (LSM, kPa) (±SD) was determined by transient elastography (TE) or shear-wave elastography (SW) in NASH (n=50) vs. non-NASH (n=34) subjects and diabetes (n=51) vs non-diabetes (n=33) subjects, as shown in FIG. 4. Percent of subjects exhibiting <20 kPa LSM, percent of fibrosis score F4 subjects (advanced liver scarring cirrhosis) (highest medical need population), and avg. CAP score (±SD) are shown in FIG. 4. Liver stiffness (LSM, kPa) and CAP scores were similar in NASH vs. non-NASH and Diabetes vs. No Diabetes subjects.

Endoscopic findings for 270 subjects by EGD were compared between NASH vs. non-NASH subjects and diabetes vs. no diabetes subjects. Table 5 shows numbers of NASH vs. non-NASH and diabetes vs. non-diabetes subjects, numbers of subjects having any varices, esophageal small varices, large varices, red wale sign, gastric varices, any portal hypertensive gastropathy (PHG), moderate to severe PHG, and endoscopic evidence for significant portal hypertension (increase in pressure within the portal vein). Endoscopic findings were similar between NASH vs. non-NASH and diabetes vs. no diabetes subjects.

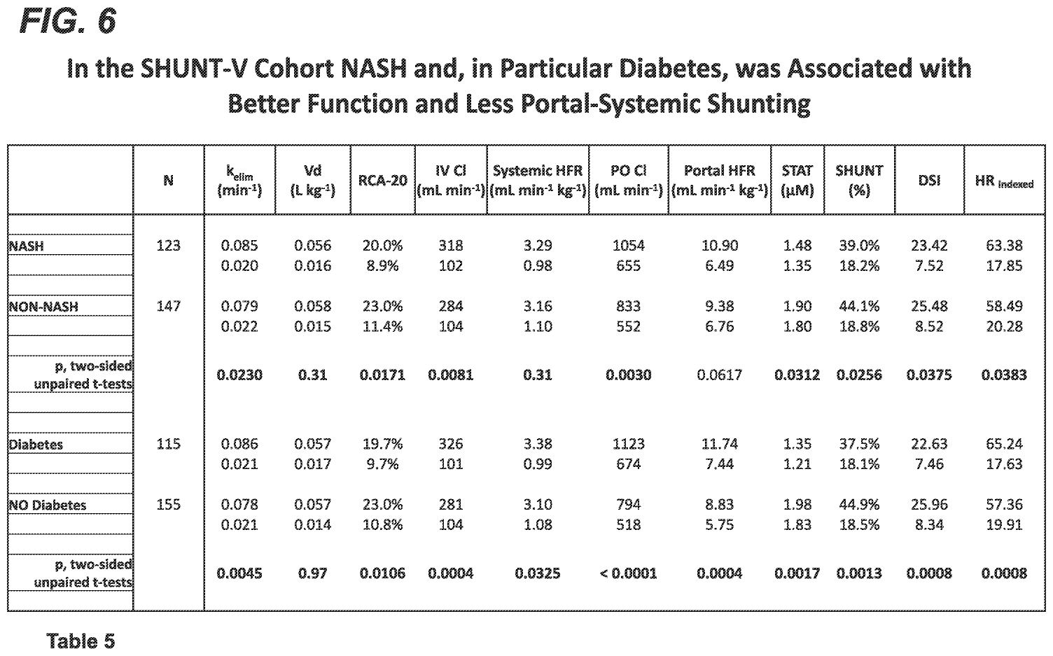

Cholate liver function test values were determined for the 270 subjects and compared between NASH vs. non-NASH subjects and diabetes vs. no diabetes subjects as shown in FIG. 6. Mean values (±SD) for cholate elimination rate, kelim (min⁻¹), cholate volume of distribution (Vd), (L kg-1), RCA20, IV Cl (ml min⁻¹), systemic HFR (ml min⁻¹ kg⁻¹), PO Cl (mL min-1), portal HFR (mL min-1 kg-1), STAT (uM), SHUNT (%), DSI, and HRindexed for NASH vs. non-NASH subjects and diabetes vs. no diabetes subjects are shown in Table 6.

Surprisingly, in the SHUNT-V cohort, NASH and especially Diabetes Mellitus were associated with better liver function and less portal-systemic shunting, compared to the non-NASH and No diabetes subjects, as shown in Table 6. NASH and Diabetic subjects exhibited significantly higher mean cholate kₑₗᵢₘ, IV Cl, PO Cl, and HR indexed than non-NASH and no diabetes subjects respectively. Diabetic subjects exhibited significantly higher mean portal HFR than no diabetes subjects. NASH and Diabetic subjects exhibited significantly lower mean cholate SHUNT %, STAT, RCA20, and DSI values than non-NASH and no diabetes subjects respectively.

Mean systemic HFR was significantly higher in diabetes subjects compared to non-diabetes subjects, as shown in FIG. 7B (p=0.0325).

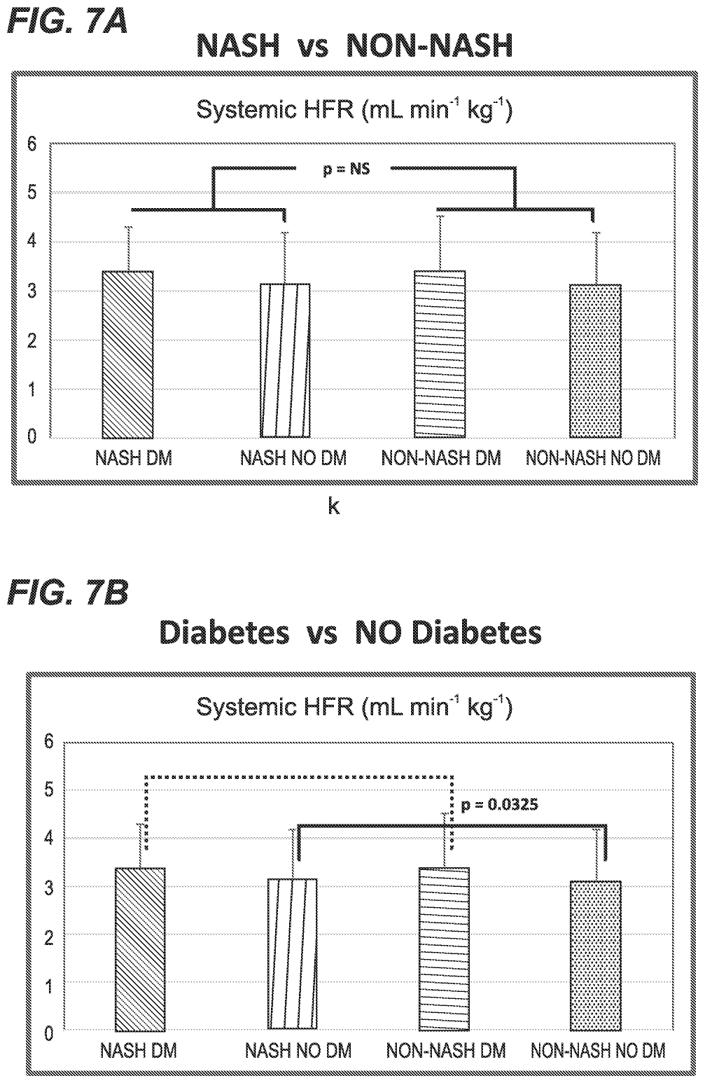

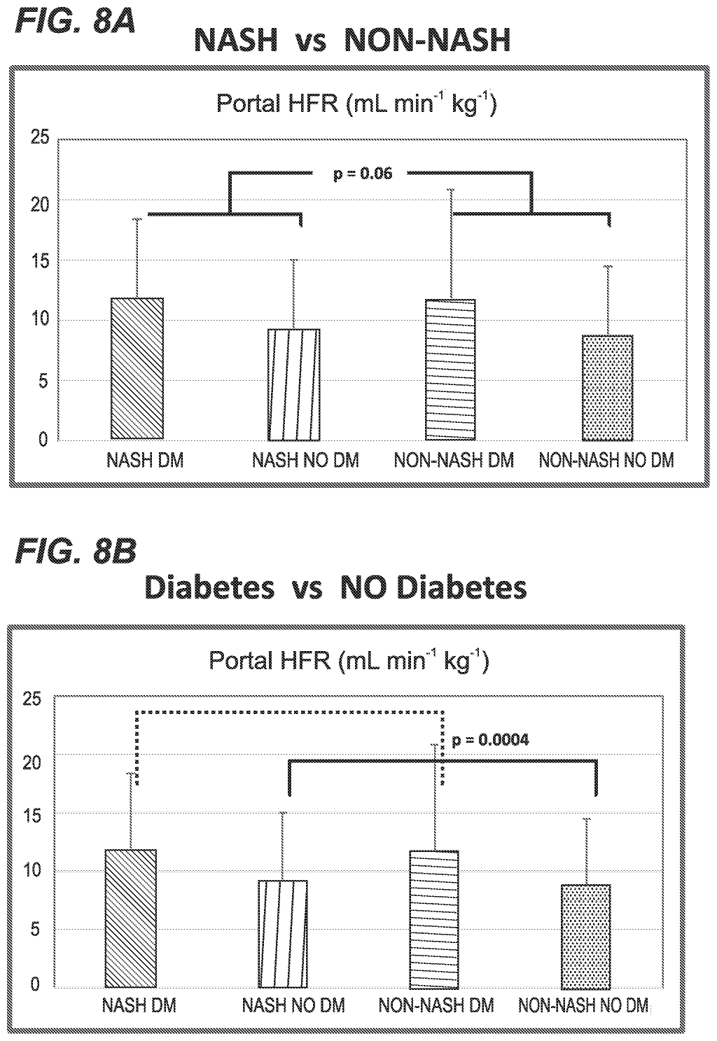

Mean portal HFR trended higher in NASH vs. non-NASH subjects, as shown in FIG. 8A (p=0.06).

Mean portal HFR was significantly higher in diabetes subjects vs. no diabetes subjects, as shown in FIG. 8B (p=0.0004).

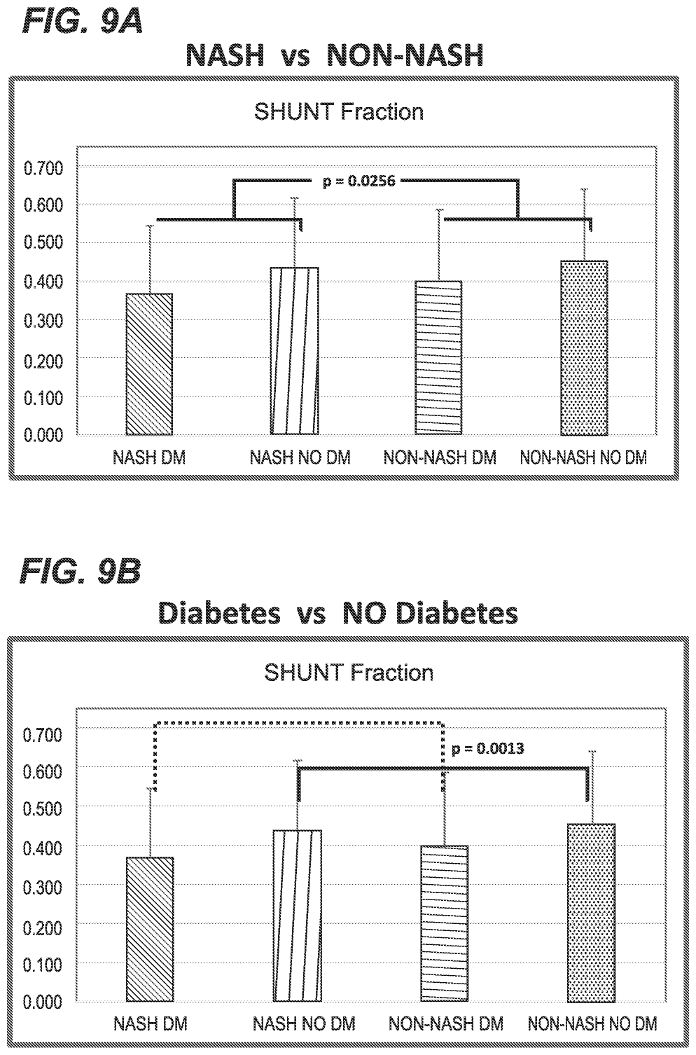

Mean SHUNT fraction was significantly lower in NASH vs. non-NASH subjects, as shown in FIG. 9A (p=0.0256).

Mean SHUNT fraction was significantly lower in diabetes subjects vs. no diabetes subjects, as shown in FIG. 9B (p=0.0013).

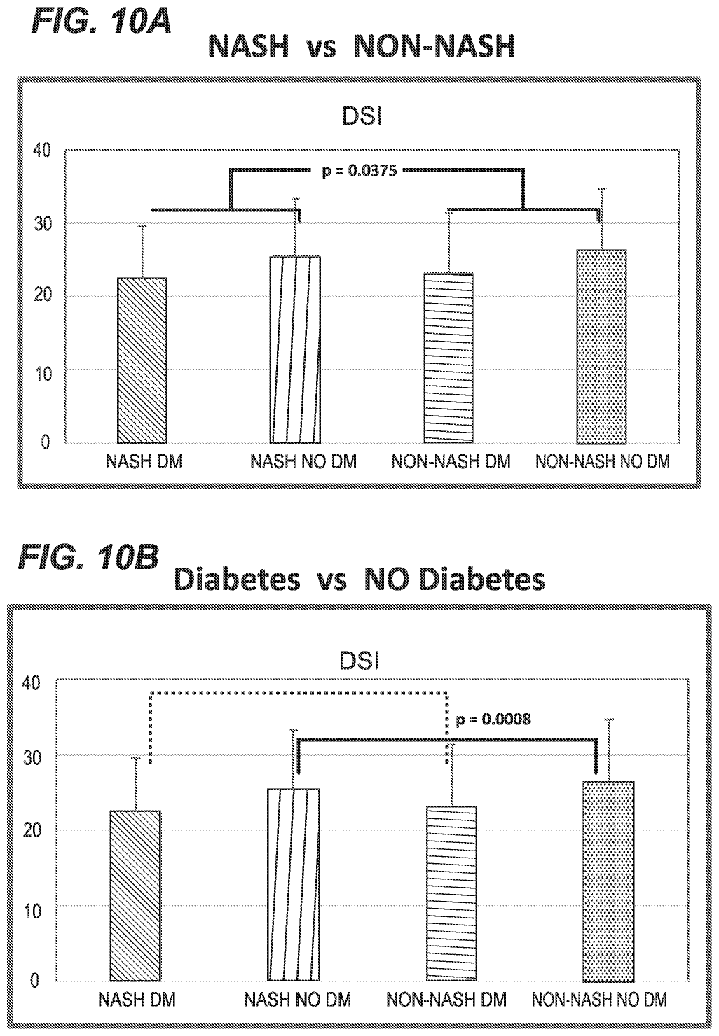

Mean DSI value was significantly lower in NASH vs. non-NASH subjects, as shown in FIG. 10A (p=0.0375).

Mean DSI value was significantly lower in diabetes subjects vs. no diabetes subjects, as shown in FIG. 10B (p=0.0008).

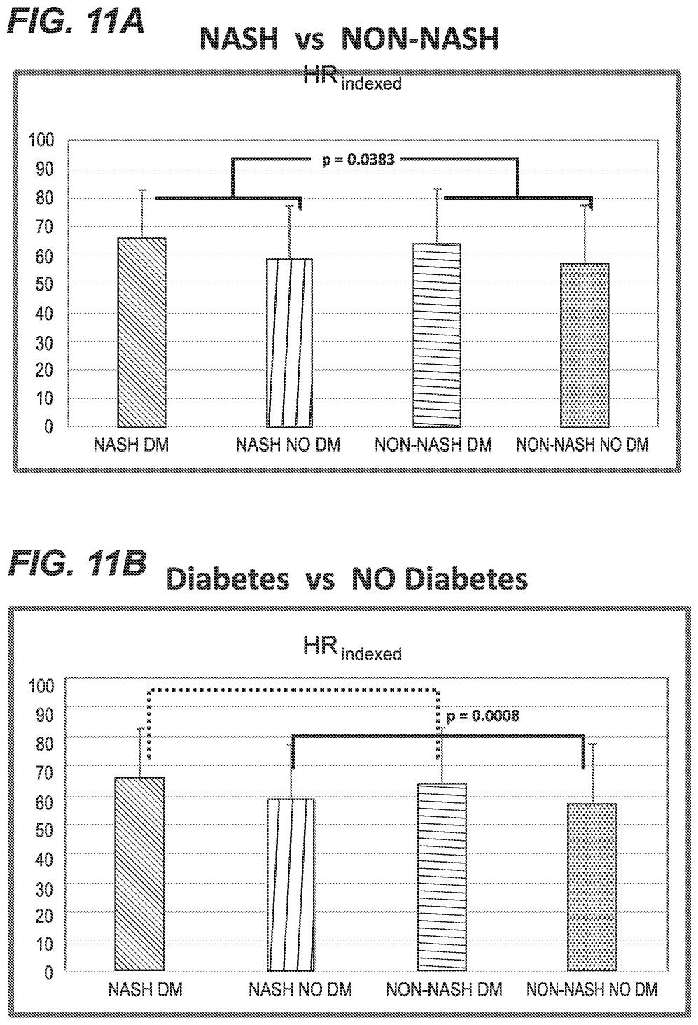

Mean indexed Hepatic Reserve (HRindexed) was significantly higher in NASH vs. non-NASH subjects, as shown in FIG. 11A (p=0.0383).

Mean indexed Hepatic Reserve (HRindexed) was significantly higher in diabetes subjects vs. no diabetes subjects, as shown in FIG. 11B (p=0.0008).

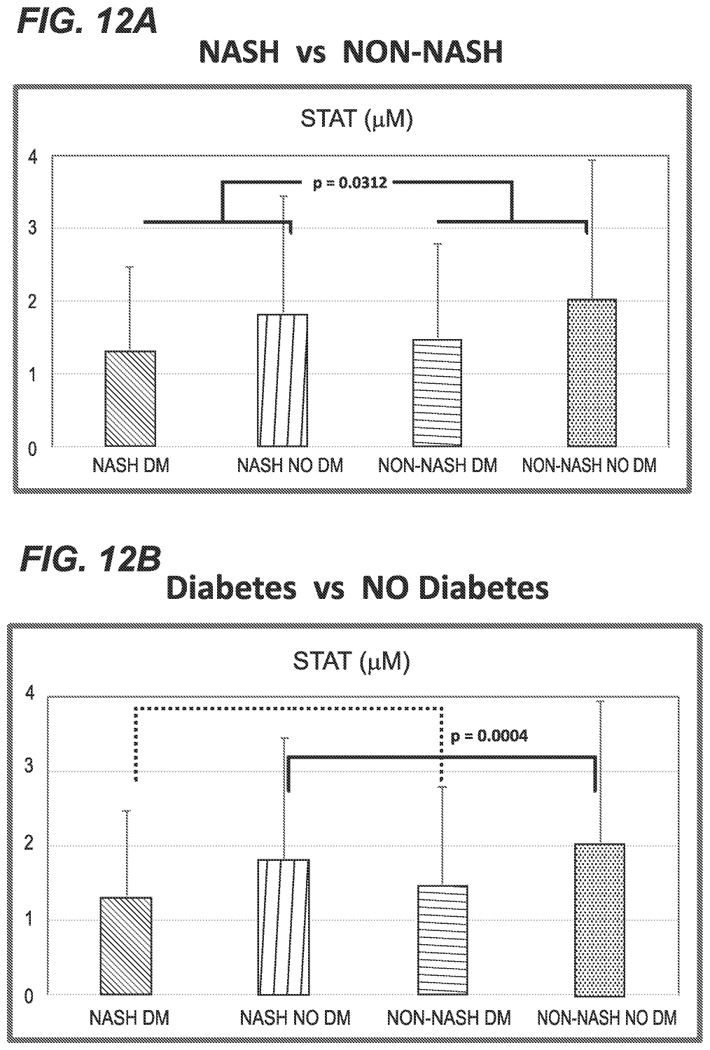

Mean cholate STAT value was significantly lower in NASH vs. non-NASH subjects, as shown in FIG. 12A (p=0.0312).

Mean cholate STAT value was significantly lower in diabetes subjects vs. no diabetes subjects, as shown in Table 5, FIG. 6 (p=0.0017), and in NASH no DM compared to non-NASH no DM subjects, as shown in FIG. 12B (p=0.0004).

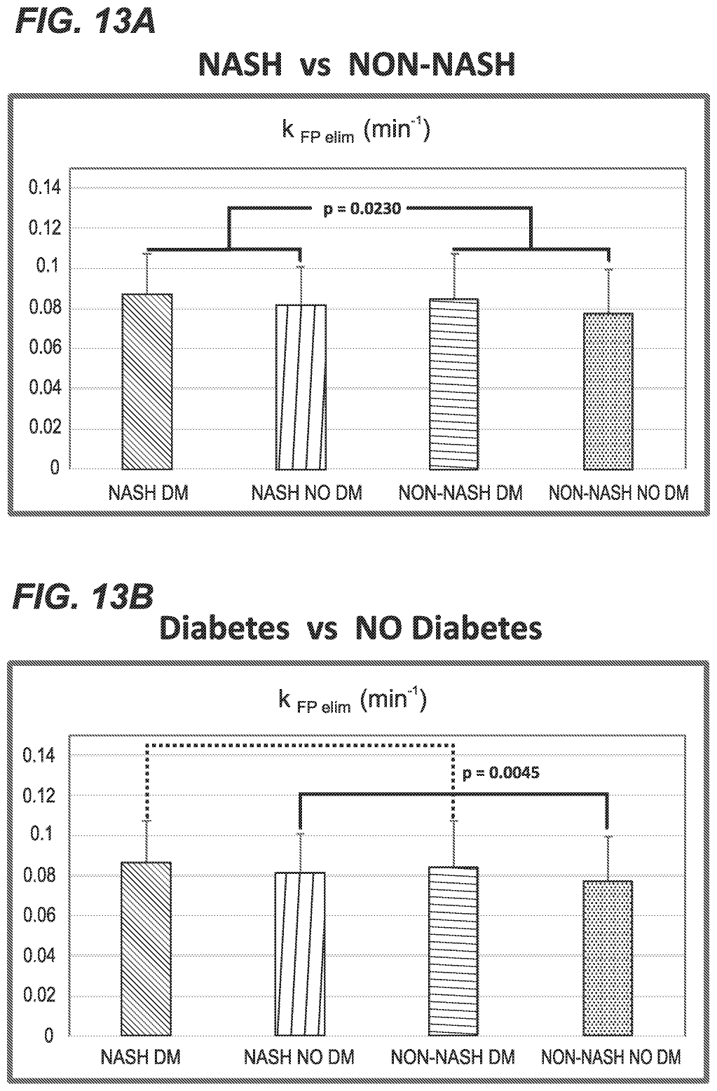

Mean Cholate Elimination Rate, kₑₗᵢₘ min⁻¹(kFP elim; min⁻¹) was significantly higher in NASH vs. non-NASH subjects, as shown in FIG. 13A (p=0.0230).

Mean Cholate Elimination Rate, kₑₗᵢₘ min⁻¹(kFP elim; min⁻¹) was significantly higher in diabetes subjects vs. no diabetes subjects, as shown in FIG. 13B (p=0.0045).

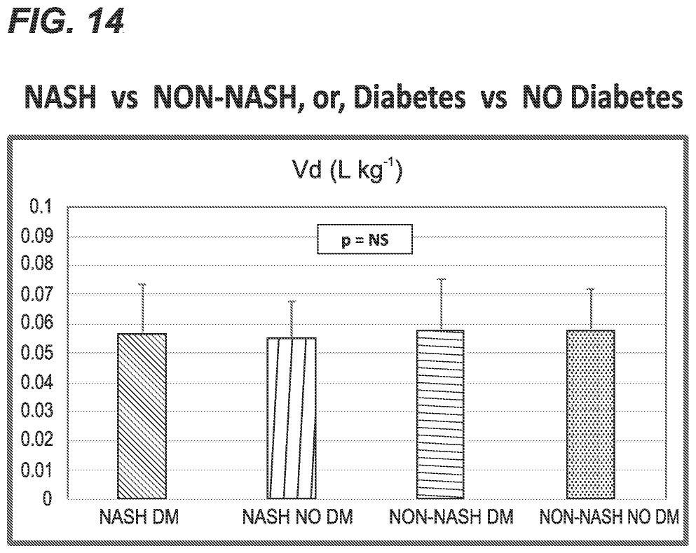

Mean cholate volume of distribution (Vd; L kg⁻¹) was not significantly different between NASH vs. non-NASH subjects or between diabetes vs. no diabetes subjects, as shown in FIG. 14.

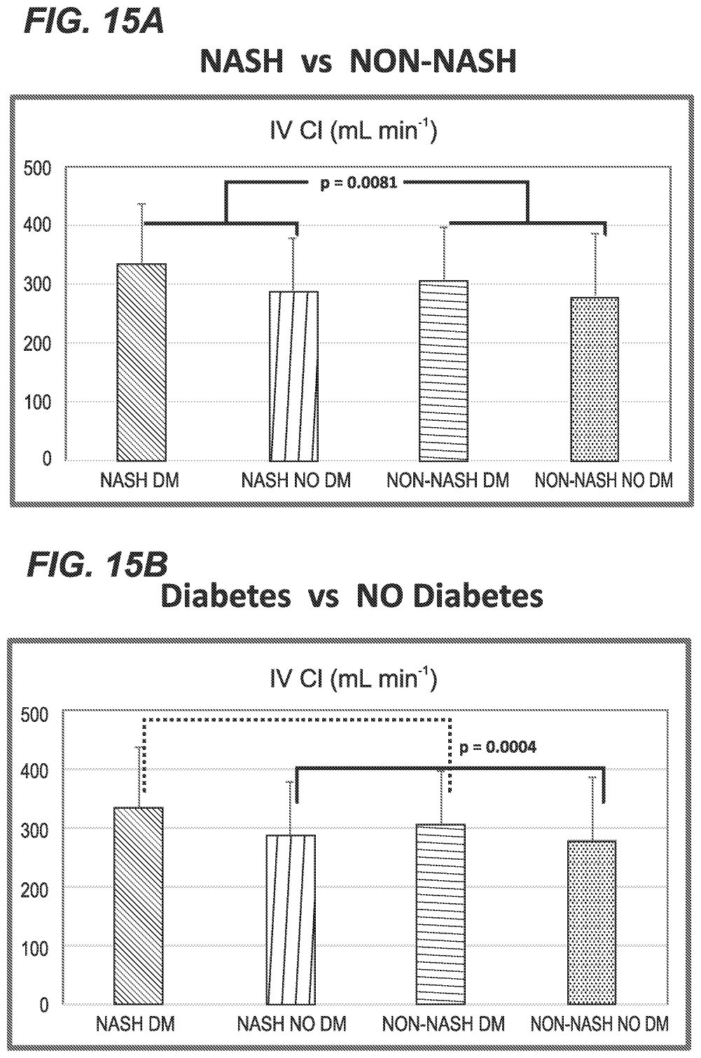

Mean intravenous cholate clearance (IV Cl) (mL min⁻¹) was significantly higher in NASH vs. non-NASH subjects, as shown in FIG. 15A (p=0.0081).

Mean intravenous cholate clearance (IV Cl) (mL min⁻¹) was significantly higher in diabetes subjects vs. no diabetes subjects, as shown in FIG. 15B (p=0.0004).

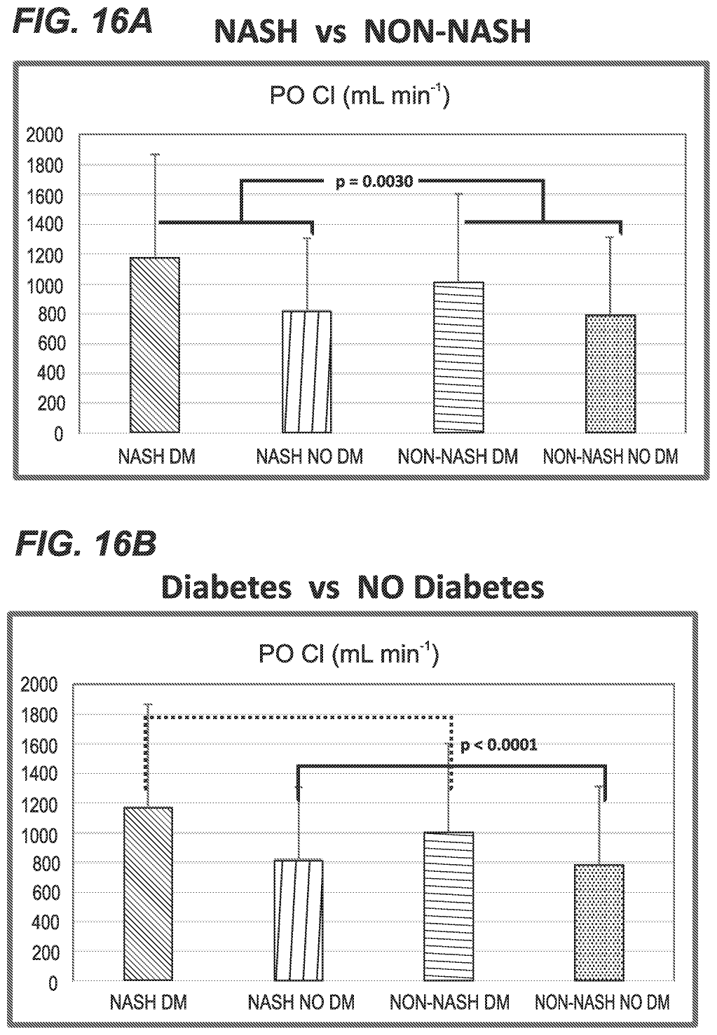

Mean oral cholate clearance (PO Cl) (mL min⁻¹) was significantly higher in NASH vs. non-NASH subjects, as shown in FIG. 16A (p=0.0030).

Mean oral cholate clearance (PO Cl) (mL min⁻¹) was significantly higher in diabetes subjects vs. no diabetes subjects, as shown in FIG. 16B (p<0.0001).

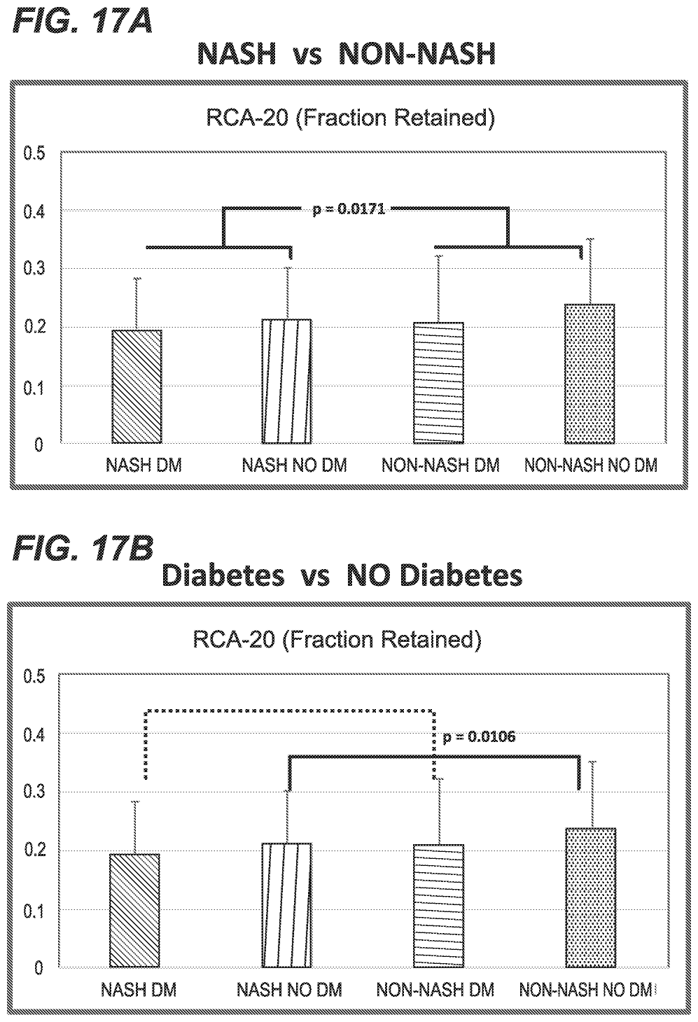

Mean RCA-20 (fraction retained) (%) was significantly lower in NASH vs. non-NASH subjects, as shown in FIG. 17A (p=0.0171).

Mean RCA-20 (fraction retained) (%) was significantly lower in diabetes subjects vs. no diabetes subjects, as shown in FIG. 17B (p=0.0106).

As shown in FIGS. 6, 7B, 8B-13B, and 15A-17B, NASH and Diabetes subjects exhibited better liver function and less portal-systemic shunting in the cholate liver function tests compared to the non-NASH and no diabetes subjects.

In order to further understand these surprising results, medications of 130 subjects taking anti-diabetic drug therapies and 140 subjects not taking antidiabetic anti-diabetic drug therapies were further investigated. DSI values and SHUNT % results relative to concomitant medications are shown in FIG. 18, Table 6.

In subjects taking the biguanide metformin, the mean difference in DSI was −2.4 (p=0.08) and the mean difference in SHUNT was −7.4% (p=0.025) indicating a trend of better liver function and decreased portal-systemic shunting.

Surprisingly, in subjects taking both anti-diabetic drug therapies and a statin, the mean difference in DSI was −3.6 (p=0.0044) and the mean difference in SHUNT was −7.6% (p=0.0141) indicating significantly better liver function and decreased portal-systemic shunting.

In a subset of 140 subjects not taking anti-diabetic drug therapies, 25 were taking statins and 115 were not taking statins. As shown in the last row of Table 6, FIG. 18, subjects taking statins not taking antidiabetic therapy, exhibited a mean difference in DSI of −3.2 (p=0.08) and a mean difference in SHUNT % or −6.6 (p=0.12).

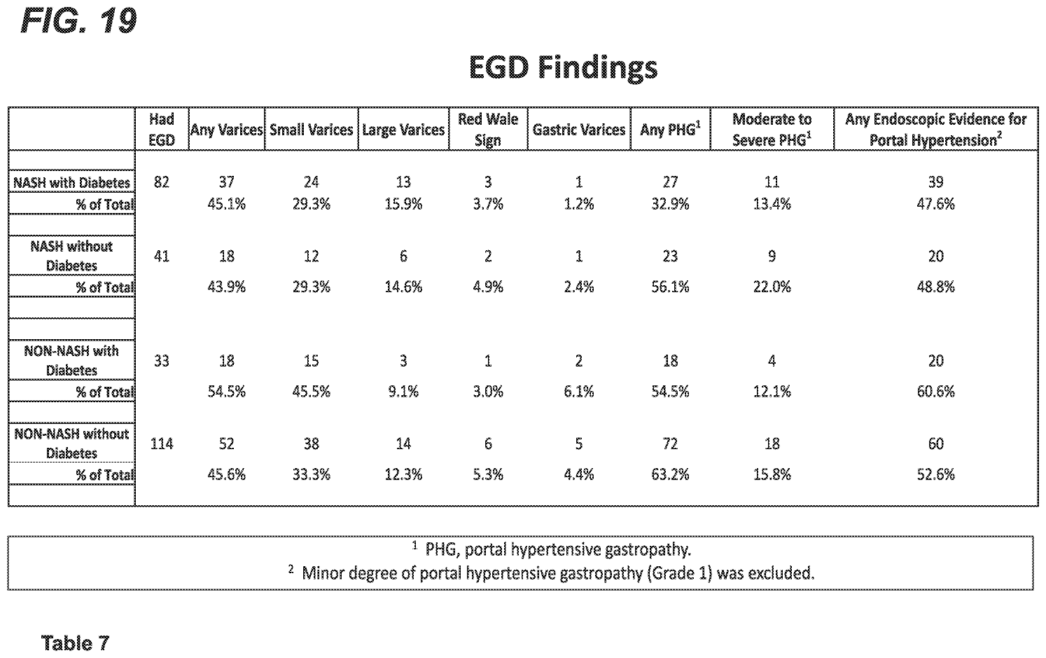

Analysis of NASH and diabetes combinations with respect to EGD findings, HepQuant SHUNT test results, and elastography within one year of enrollment are shown in FIGS. 19, 20 and 21 respectively.

EGD findings for subjects having NASH with diabetes (n=82), NASH without diabetes (n=41), Non-NASH with diabetes (n=33), and Non-NASH without diabetes (n=114) are shown in FIG. 19, Table 7. Number of subjects in each group having any varices, esophageal small varices, large varices, red wale sign, gastric varices, any portal hypertensive gastropathy (PHG), moderate to severe PHG, and endoscopic evidence for significant portal hypertension (increase in pressure within the portal vein). Endoscopic findings were similar between NASH vs. non-NASH and diabetes vs. no diabetes subjects.

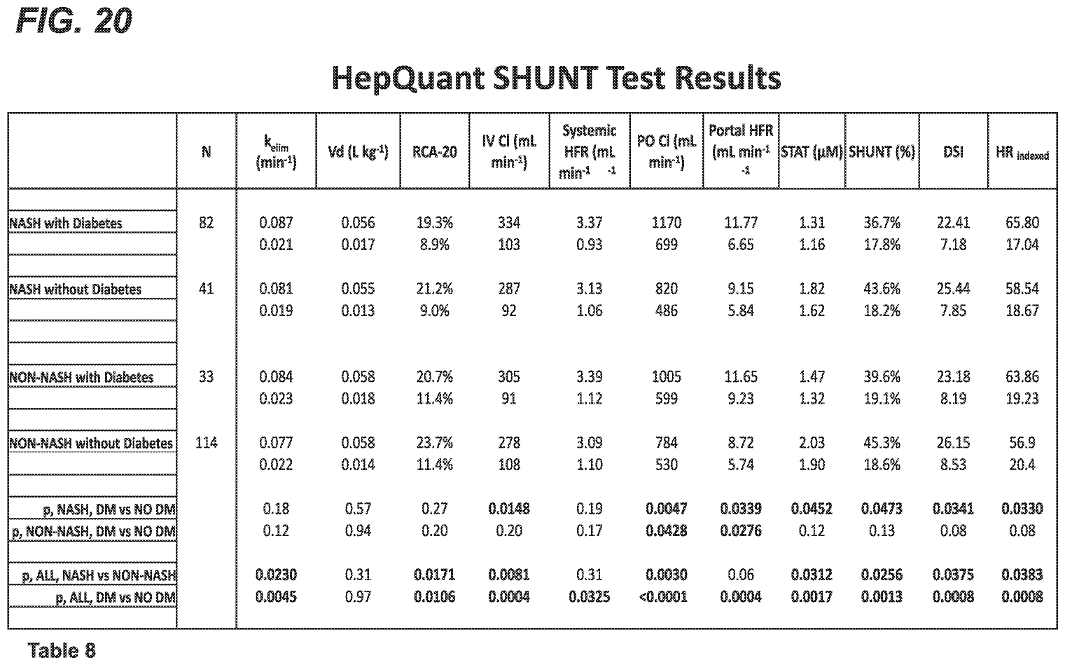

HepQuant cholate liver function test results for subjects having NASH with diabetes (n=82), NASH without diabetes (n=41), Non-NASH with diabetes (n=33), and Non-NASH without diabetes (n=114) are shown in FIG. 20, Table 8.

For subjects having NASH, with diabetes vs. no diabetes, IV CL was significantly higher (p=0.0148), PO CL was significantly higher (p=0.0047), portal HFR was significantly higher (p=0.0339), STAT was significantly lower (p=0.0452), SHUNT was significantly lower (p=0.0473), DSI was significantly lower (p=0.0341), and HRindexed was significantly higher (p=0.0330), as shown in Table 8.

For non-NASH subjects, with diabetes vs. no diabetes, PO CL was significantly higher (p=0.0428), and portal HFR was significantly higher (p=0.0276), as shown in Table 8.

For all subjects, with NASH vs. non-NASH subjects, kelim was significantly higher (p=0.0230), RCA-20 was significantly lower (p=0.0171), IV Cl was significantly higher (p=0.0081), PO Cl was significantly higher (p=0.0030), STAT was significantly lower (p=0.0312), SHUNT % was significantly lower (p=0.0256), DSI was significantly lower (p=0.0375), and HRindexed was significantly higher (p=0.0383), as shown in Table 8.

For all subjects, with diabetes vs. no diabetes, kelim was significantly higher (p=0.0045), RCA-20 was significantly lower (p=0.0106), IV Cl was significantly higher (p=0.0004), PO Cl was significantly higher (p=0.0000), STAT was significantly lower (p=0.0017), SHUNT % was significantly lower (p=0.0013), DSI was significantly lower (p=0.0008), and HRindexed was significantly higher (p=0.0008), as shown in Table 8.

As shown in Table 8, FIG. 20, surprisingly NASH and Diabetes subjects exhibited significantly better liver function and less portal-systemic shunting in the cholate liver function tests compared to the non-NASH and no diabetes subjects.

Elastography within 1 year of enrollment was performed in 53 of 123 NASH subjects (43%) and 36 of 147 non-NASH subjects.

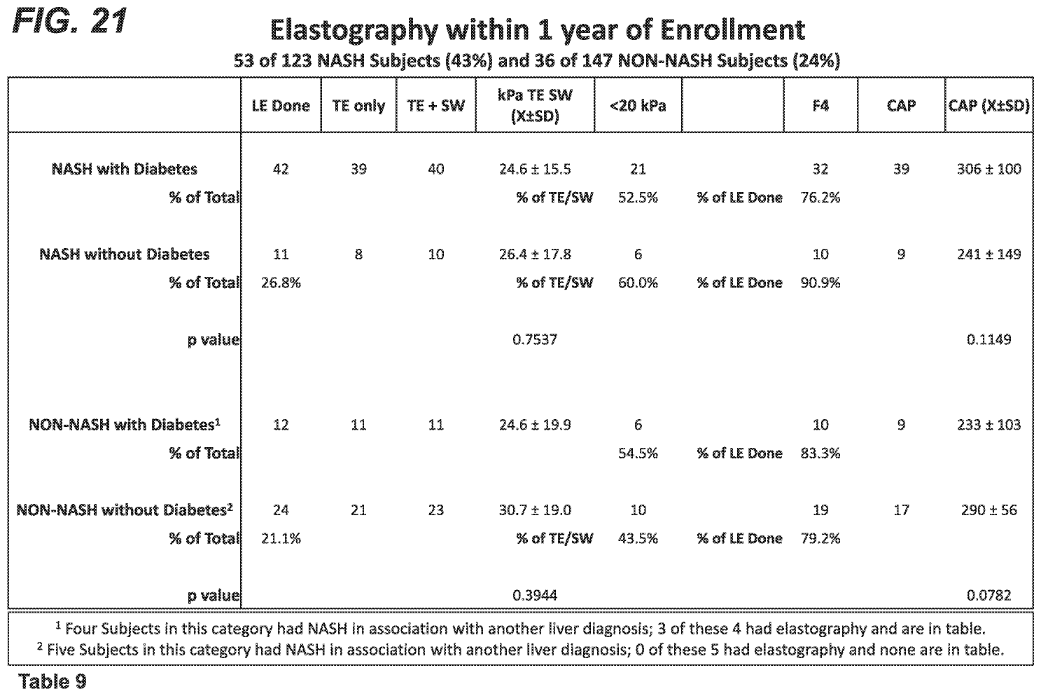

Elastography results for subjects having NASH with diabetes (n=42), NASH without diabetes (n=11), Non-NASH with diabetes (n=12), and Non-NASH without diabetes (n=24) are shown in FIG. 21, Table 9. CAP score for non-NASH subjects, with diabetes vs. no diabetes exhibited a trend to lower CAP scores (p=0.0782). A lower CAP score indicates a lower amount of hepatic steatosis, or lower amount of liver with fatty change.

### Example 2. HMG-CoA Reductase Inhibitors (Statins) and Metformin are Associated with Preservation of Hepatic Function and Less Portal-Systemic Shunting in Advanced Chronic Liver Disease (CLD)

Background: Disease progression of CLD and nonalcoholic steatohepatitis (NASH) is variable and may be influenced by comorbid conditions and concomitant drug therapy. We used the HepQuant SHUNT test to evaluate the impact of NASH, diabetes mellitus (DM), and drug therapy on hepatic function and portal-systemic shunting in subjects with CLD enrolled in SHUNT-V study.

Methods: The 270 subjects in the preliminary analysis had either compensated cirrhosis, fibrosis stage F3 with platelet count <175,000, or a Child-Pugh B without refractory ascites, refractory encephalopathy, or history of variceal hemorrhage. HepQuant tests involved dosing [24-¹³C-cholic acid, IV, and [2,2,4,4-²H]-cholic acid, PO, and blood sampling at t=0, 5, 20, 45, 60, and 90 minutes. Serum was analyzed for cholate concentrations by LC-MS/MS and a disease severity index (DSI), assessing hepatic function, and portal-systemic shunt fraction (SHUNT) were calculated. Lower DSI indicates better hepatic function; lower SHUNT indicates less portal-hepatic shunting.

Results: Subject characteristics (means or percentages): age 61.6 years, body weight 95.3 kg, BMI 33.4, male 49%, white race 92%, Hispanic ethnicity 14%; 64% were obese, 50% had NASH, and 48% were taking diabetic drug therapy. Compared to other etiologies for CLD, NASH subjects were older, heavier, and had higher BMI and more were obese. In contrast, NASH and non-NASH subjects had similar blood tests, clinical scores, elastography and endoscopic findings.

Table 12 shows a comparison of mean HepQuant cholate test values for Systemic HFR (mL min⁻¹ kg⁻¹), Portal HFR (mL min⁻¹ kg⁻¹), SHUNT (%), and DSI score in NASH subjects (n=123) compared to non-NASH subjects (n=147). Surprisingly, NASH subjects exhibited better liver function (lower DSI scores) and less portal-hepatic shunting (lower SHUNT %).

TABLE 12
Cholate Test Scores in NASH vs non-NASH Subjects

| | | Systemic HFR | Portal HFR | | |
|---|---|---|---|---|---|
| | | mL min⁻¹ | mL min⁻¹ | SHUNT | DSI |
| | | kg⁻¹ | kg⁻¹ | % | Score |
| NASH | N | 123 | 123 | 123 | 123 |
| | Mean | 3.29 | 10.90 | 39.0% | 23.4 |
| | SD | 0.98 | 6.49 | 18.2% | 7.5 |
| Non- | N | 147 | 147 | 147 | 147 |
| NASH | Mean | 3.16 | 9.38 | 44.1% | 25.5 |
| | SD | 1.10 | 6.76 | 18.8% | 8.5 |
| t-test | p | 0.31 | 0.0617 | 0.0256 | 0.0375 |

Table 13 shows a comparison of mean HepQuant cholate test values for Systemic HFR (mL min⁻¹ kg⁻¹), Portal HFR (mL min⁻¹ kg⁻¹), SHUNT (%), and DSI score in diabetes mellitus (DM) subjects (n=115) compared to non-DM subjects (n=155). Surprisingly, diabetes (DM) subjects exhibited better liver function (lower DSI scores) and less portal-hepatic shunting (lower SHUNT %).

TABLE 13
Cholate Test Scores in diabetes (DM)
vs non-diabetes (non-DM) Subjects

| | | Systemic HFR | Portal HFR | | |
|---|---|---|---|---|---|
| | | mL min−1 | mL min−1 | SHUNT | DSI |
| | | kg−1 | kg−1 | % | Score |
| DM | N | 115 | 115 | 115 | 115 |
| | Mean | 3.38 | 11.74 | 37.5% | 22.63 |
| | SD | 0.99 | 7.44 | 18.1% | 7.46 |
| No-DM | N | 155 | 155 | 155 | 155 |
| | Mean | 3.10 | 8.83 | 44.9% | 26.0 |
| | SD | 1.08 | 5.75 | 18.5% | 8.3 |
| | P | 0.0325 | 0.0004 | 0.0013 | 0.0008 |

Subjects were stratified as to drug therapy regimens. In univariable regression, diabetic drug therapy and statins were associated with lower DSI and lower SHUNT %. In univariable regression analysis, the diabetes drugs metformin, glipizide/glimepiride, SGLT2 inhibitors, and insulin were each associated with significantly reduced DSI scores (improved liver function) as shown in Table 14. Lipid lowering drugs and in particular statins were also associated with significantly reduced DSI scores (improved liver function).

TABLE 14
Univariable Regression Analysis of Diabetic
Drug Therapy and Statins on Δ DSI score

| | | ΔDSI | SE | 95% CI | t | p |
|---|---|---|---|---|---|---|
| Diabetes Drugs | 130 | −3.9588 | 0.9621 | −5.8530 to −2.0645 | −4.1147 | 0.0001 |
| Metformin | 89 | −4.1777 | 1.0231 | −6.1921 to −2.1634 | −4.0833 | 0.0001 |
| Glipizide/Glimepiride | 46 | −4.0032 | 1.2956 | −6.5540 to −1.4525 | −3.0900 | 0.0022 |
| SGLT2 Inhibitors | 34 | −3.1089 | 1.4819 | −6.0266 to −0.1911 | −2.0978 | 0.0369 |
| Insulin | 50 | −2.746 | 1.265 | −5.2365 to −0.2554 | −2.1708 | 0.0308 |
| Pioglitazone | 14 | −2.4995 | 2.303 | −6.8907 to 1.8916 | −1.1207 | 0.2634 |
| GLP-1 Analogue | 36 | −1.5678 | 1.455 | −4.4325 to 1.2969 | −1.0775 | 0.2822 |
| DPP-4 Inhibitor | 7 | −3.5953 | 3.1114 | −9.7212 to 2.5306 | −1.1555 | 0.2489 |
| Lipid-Lowering Drugs | 80 | −4.1608 | 0.9748 | −6.0801 to −2.2416 | −4.2684 | <0.0001 |
| Statin | 72 | −4.518 | 0.9956 | −6.4781 to −2.5578 | −4.5380 | <0.0001 |
| Vitamin E | 14 | −2.0475 | 2.232 | −6.4420 to 2.3470 | −0.9173 | 0.3598 |

Multi-variable regression analyses of prescribed medications and mean A DSI scores compared to base DSI scores are shown in Table 15.

In multi-variable regression analysis of prescribed medications, statins and metformin exhibited the strongest associations with lower DSI (p=0.003, p=0.06, respectively) and SHUNT (p<0.015, p<0.85, respectively, data not shown).

TABLE 15
Multi-variable Regression Analysis of Diabetic Drugs
and Statins on Cholate Liver Function Δ DSI score

| | | | | | % |
|---|---|---|---|---|---|
| Independent | N On | Coeffi- | | Resultant | Reduction |
| variables | Drug | cient | P | DSI | in DSI |
| Base DSI | — | 27.0682 | | | |
| Statins | 72 | −3.2543 | 0.0029 | 23.81 | −12.0% |
| Metformin | 89 | −2.2637 | 0.0638 | 21.55 | −20.4% |
| Glipizide/Glimepiride | 46 | −1.9393 | 0.18 | — | — |
| GLP_1_Analogue | 36 | 1.0743 | 0.50 | — | — |
| Pioglitazone | 14 | 0.2725 | 0.91 | — | — |
| SGLT-2_Inhib | 34 | −1.1805 | 0.47 | — | — |
| DPP_4_Inhib | 7 | −2.1736 | 0.48 | — | — |
| INSULIN | 50 | −0.7947 | 0.55 | — | — |
| Vitamin E | 14 | −1.6505 | 0.45 | — | — |

Multi-variable regression including statins, DM, NASH, and diabetic drug therapy are shown in Table 16. Statins were the predominant factor associated with both lower DSI and lower SHUNT %.

TABLE 16
Multi-variable Regression Analysis of Statins,
Metformin, diabetes diagnosis, and NASH diagnosis
on decline in SHUNT % and DSI scores

| | Impact on SHUNT % | | Impact on DSI | |
|---|---|---|---|---|
| | Decline in | | Decline in | |
| | SHUNT % | p | DSI | p |
| Statin | −6.3% | 0.0132 | −3.3269 | 0.0025 |
| Metformin | −5.9% | 0.0475 | −2.4337 | 0.0574 |
| Diabetes Diagnosis | −1.4% | 0.64 | −0.7239 | 0.5736 |
| NASH Diagnosis | −1.3% | 0.61 | −0.2246 | 0.8343 |

The estimated effect of combined treatment with statins and diabetes drugs was a greater than 20% lowering of both DSI score and SHUNT %.

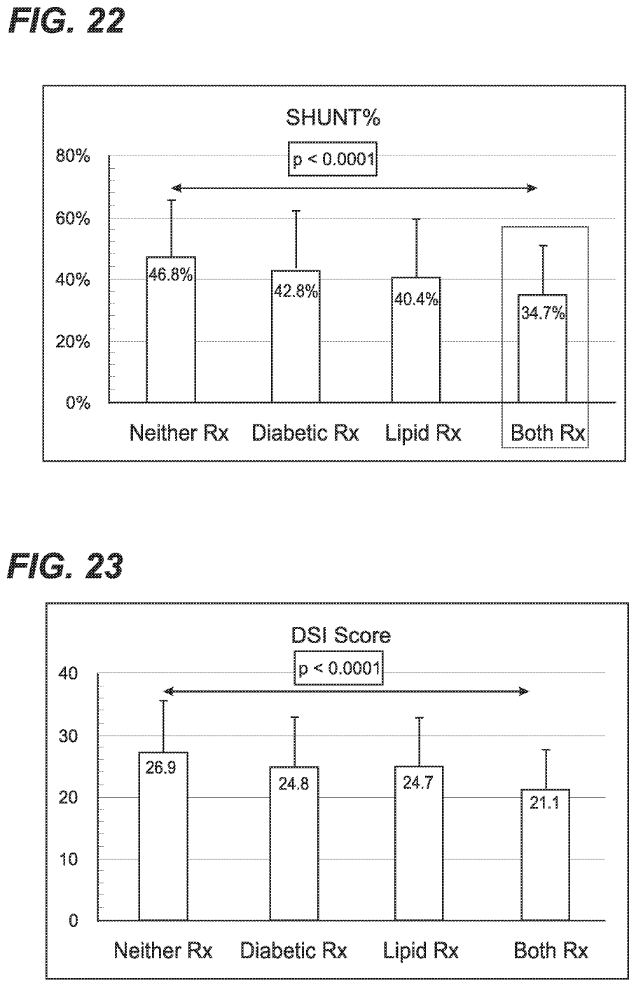

A bar graph of the effect of diabetic and/or lipid lowering drugs on mean SHUNT % score is shown in FIG. 22. Diabetic and lipid-lowering drugs use is associated with less portal-systemic shunting (lower SHUNT %). *p<0.0001 for change from treatment with neither drug (left hand bar) to treatment with both classes of drug (right hand bar).

A bar graph of the effect of diabetic and/or lipid lowering drugs on mean DSI score is show in FIG. 23. Diabetic and lipid-lowering drugs use is associated with better liver function (lower DSI). *p<0.0001 for change from treatment with neither drug (left hand bar) to treatment with both classes of drug (right hand bar).

What is claimed is:

1. A method of treating or preventing a chronic liver disease or disorder in a subject comprising:
a) determining a liver function test value in the subject;
b) comparing the liver function test value to a predetermined cutoff value; and
c) administering to the subject a therapeutically effective amount of a statin or a pharmaceutically acceptable salt thereof, and coadministering a therapeutically effective amount of a biguanide or a pharmaceutically acceptable salt thereof, when the liver function test value is equal to or greater than the predetermined cutoff value.

2. A method of treating or preventing a chronic liver disease or disorder in a subject comprising:
a) determining a first liver function test value in the subject at a time point prior to the administration of a therapeutically effective amount of a statin or a pharmaceutically acceptable salt thereof, and coadministering a therapeutically effective amount of a biguanide or a pharmaceutically acceptable salt thereof;
b) administering to the subject a therapeutically effective amount of a statin or a pharmaceutically acceptable salt thereof, and a therapeutically effective amount of a biguanide or a pharmaceutically acceptable salt thereof;
c) determining at least a second liver function test value in the subject at least one time point after the administration of the therapeutically effective amount of a statin or a pharmaceutically acceptable salt thereof, and a therapeutically effective amount of a biguanide or a pharmaceutically acceptable salt thereof;
d) determining a liver function test difference score by calculating the difference between the at least second liver function test value and the first liver function test value; and
e) comparing the liver function test difference score to a predetermined cutoff value.

3. The method of claim 2, further comprising:
f) discontinuing or decreasing administration of the statin or a pharmaceutically acceptable salt thereof, and coadministration of the biguanide or a pharmaceutically acceptable salt thereof when the liver function difference score is less than or equal to the predetermined cutoff value, or continuing administration of the statin or a pharmaceutically acceptable salt thereof, and the biguanide or a pharmaceutically acceptable salt thereof when the liver function difference score is greater than or equal to the predetermined cutoff value.

4. The method of claim 1, wherein the chronic liver disease or disorder is selected from the group consisting of non-alcoholic steatohepatitis (NASH), non-alcoholic fatty liver disease (NAFLD), alcoholic liver disease, drug-induced liver disease, chronic hepatitis C, chronic hepatitis B, cytomegalovirus, Epstein Barr virus, portal hypertension, cryptogenic cirrhosis, alpha 1-antitrypsin disease, hemochromatosis, nodular regenerative hyperplasia, idiopathic liver disease, congenital liver diseases, haemochromatosis, Wilson's disease, autoimmune chronic hepatitis, primary biliary cirrhosis, primary sclerosing cholangitis (PSC), liver damage due to progressive fibrosis, liver fibrosis, and hepatocellular carcinoma (HCC).

5. The method of claim 1, wherein the subject suffers from diabetes mellitus (DM).

6. The method of claim 1, wherein the liver function test is a cholate liver function test.

7. The method of claim 6, wherein the cholate liver function test is selected from the group consisting of cholate SHUNT fraction, DSI value, portal hepatic filtration rate (portal HFR)(mL min⁻¹kg⁻¹), systemic hepatic filtration rate (systemic HFR)(mL min⁻¹kg⁻¹), indexed Hepatic Reserve (HRindex), STAT value (uM), k_{FP elim} (min⁻¹), IV clearance (mL min⁻¹), PO clearance (ml min⁻¹), and RCA-20 (fraction retained).

8. The method of claim 1, wherein the statin is selected from the group consisting of lovastatin, pravastatin, simvastatin, fluvastatin, cerivastatin, atorvastatin, pitavastatin, and rosuvastatin.

9. The method of claim 8, wherein the daily dose of the statin is in a range from about 5 mg to about 100 mg daily, about 10 mg to about 80 mg daily, or about 20 mg to about 40 mg.

10. The method of claim 1, wherein the biguanide is metformin.

11. The method of claim 10, wherein the daily dose of the metformin is in a range from about 500 mg to about 2550 mg daily, about 800 to about 2,000 mg daily, or about 1,000 mg/daily to about 1,500 mg.

12. The method of claim 1, wherein the method further comprises coadministration of an additional agent selected from the group consisting of vitamin E, ursodeoxycholic acid, sulfonylurea, and PPAR-gamma agonist.

13. A method of treating a chronic liver disease in a subject comprising administering a statin and a biguanide.

14. The method of claim 13, wherein the chronic liver disease is selected from the group consisting of nonalcoholic fatty liver disease (NAFLD), nonalcoholic steatohepatitis (NASH), hepatitis B infection, hepatitis C infection, alcoholic liver disease, liver damage due to progressive fibrosis, or liver fibrosis.

15. The method of claim 14, wherein the liver disease is NASH.

16. The method of claim 14, wherein the subject suffers from diabetes mellitus (DM).

17. The method of claim 13, wherein the statin is selected from the group consisting of lovastatin, pravastatin, simvastatin, fluvastatin, cerivastatin, atorvastatin, pitavastatin, and rosuvastatin.

18. The method of claim 17, wherein the daily dose of the statin is in a range from about 5 mg to about 100 mg daily, about 10 mg to about 80 mg daily, or about 20 mg to about 40 mg daily.

19. The method of claim 13, wherein the biguanide is metformin.

20. The method of claim 19, wherein the daily dose of the metformin is in a range from about 500 mg to about 2550 mg daily, about 800 to about 2,000 mg daily, or about 1,000 mg/daily to about 1,500 mg daily.

21. The method of claim 13, wherein the method further comprises coadministration of an additional agent selected from the group consisting of vitamin E, ursodeoxycholic acid, sulfonylurea, and PPAR-gamma agonist.

22. A pharmaceutical composition comprising a statin or a pharmaceutically acceptable salt thereof, a biguanide or a pharmaceutically acceptable salt thereof, and a pharmaceutically acceptable carrier.

23. The pharmaceutical composition of claim 22, wherein the statin or pharmaceutically acceptable salt thereof is selected from the group consisting of lovastatin, pravastatin, simvastatin, fluvastatin, cerivastatin, atorvastatin, and rosuvastatin, or a pharmaceutically acceptable salt thereof.

24. The pharmaceutical composition of claim 23, comprising the statin or pharmaceutically acceptable salt thereof in a range of from about 5 mg to about 100 mg.

25. The pharmaceutical composition of claim 22, wherein the biguanide is metformin or a pharmaceutically acceptable salt thereof.

26. The pharmaceutical composition of claim 25, comprising the metformin or pharmaceutically acceptable salt thereof in a range from about 250 mg to about 2500 mg.

27. The pharmaceutical composition of claim 22, comprising from about 5 mg to about 100 mg of the statin or pharmaceutically acceptable salt thereof and about 250 mg to about 2500 mg daily of the biguanide or pharmaceutically acceptable salt thereof.

28. The pharmaceutical composition of claim 22, further comprising an additional agent selected from the group consisting of vitamin E, ursodeoxycholic acid, sulfonylurea, and PPAR-gamma agonist.

29. The pharmaceutical composition of claim 28, wherein the additional agent comprises vitamin E.

30. A method of treating or preventing a chronic liver disease or disorder in a subject in need thereof comprising administering the composition of claim 22.

31. The method of claim 30, wherein the chronic liver disease or disorder is selected from the group consisting of non-alcoholic steatohepatitis (NASH), non-alcoholic fatty liver disease (NAFLD), alcoholic liver disease, drug-induced liver disease, chronic hepatitis C, chronic hepatitis B, cytomegalovirus, Epstein Barr virus, portal hypertension, cryptogenic cirrhosis, alpha 1-antitrypsin disease, hemochromatosis, nodular regenerative hyperplasia, idiopathic liver disease, congenital liver diseases, haemochromatosis, Wilson's disease, autoimmune chronic hepatitis, primary biliary cirrhosis, primary sclerosing cholangitis (PSC), liver damage due to progressive fibrosis, liver fibrosis, and hepatocellular carcinoma (HCC).

32. The method of claim 31, wherein the subject suffers from diabetes mellitus (DM).
